(19) United States
(12) Patent Application Publication
(10) Pub. No.: US 2024/0294470 A1
(43) Pub. Date: Sep. 5, 2024

(54) FRAGRANCE COMPOUNDS

(71) Applicant: Givaudan SA, VERNIER (CH)

(72) Inventors: Chao WANG, Shanghai (CN); Lijun ZHOU, Shanghai (CN); Felix FLACHSMANN, Duebendorf (CH)

(21) Appl. No.: 18/569,975

(22) PCT Filed: Jun. 16, 2022
(86) PCT No.: PCT/EP2022/066505

§ 371 Date: Dec. 13, 2023

(30) Foreign Application Priority Data
Jun. 18, 2021 (WO) PCT/CN2021/100985

(51) Int. Cl. C07C 291/02; A61K 8/41; A61Q 5/02; C07C 45/42; C07D 307/52; C07D 317/58; C11B 9/00; C11D 3/30; C11D 3/50

(52) U.S. Cl. CPC C07C 291/02; A61K 8/41; A61Q 5/02; C07C 45/42; C07D 307/52; C07D 317/58; C11B 9/0007; C11D 3/30; C11D 3/50; C07C 2601/04; C07C 2601/08; C07C 2601/14; C07C 2601/16

(57) ABSTRACT

It is described the use of a compound according to formula (I)

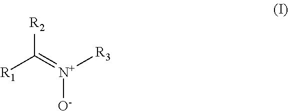

as precursor in perfumery for generating a ketone or aldehyde of formula (II)

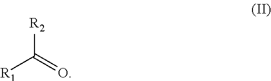

### TECHNICAL FIELD

The present invention relates generally to fragrance precursors, which are able to release fragrant carbonyl compounds like aldehydes or ketones. The invention also relates to perfume preparations and consumer products containing said precursors. The invention further relates to methods of making said perfume precursors, perfume preparations and consumer products, as well as the use of said perfume precursors and perfume preparations in consumer products, such as personal care and household care products.

### BACKGROUND

Perfumed consumer products such as cleaning or laundry products comprising fragrances are well-known in the art. However, it is known that fragrances can be altered through degradation caused by interaction with air or when incorporated in certain consumer product bases, where alkalinity, acidity, the presence of oxidizing agents, such as hypochlorite salts, or other base components may lead to chemical degradation of the fragrance. In addition, volatile fragrances tend to be dissipated with time. Furthermore, when used in cleaning or laundry products, the deposition of the fragrance on a treated substrate is diminished by the washing and/or rinsing procedure.

Nevertheless, it is desired by consumers to have products that can be stored over time and still giving a constant perfume impression. In particular, the impact of volatile components is to be retained. Furthermore, it is desired that such products create a long-lasting pleasing fragrance slowly emitting from the treated substrate overtime.

To address these needs, fragrance precursors can be used, which are substances that are essentially odorless themselves, but which, in particular circumstances, will decompose to release the fragrant molecule.

There are several classes of known precursors which release fragrant molecules upon activation, such as hydrolysis, temperature change, oxygen, action of light and enzymes. For example, WO2012085287 reports a group of precursors able to release a fragrance by spontaneous air oxidation. In WO2007143873 another group of precursors is described which can be cleaved by hydrolysis.

All precursors do show different stability, and they release the fragrant molecule under different conditions. For example, there is still need to provide further precursor systems to improve fragrance effects on dry fabric after some days. It is therefore desirable to provide a new or improved system which is capable of releasing fragrance over an extended time period and providing a long-lasting release of said fragrance.

### SUMMARY

In accordance with a first aspect of the present invention there is provided a use of a compound as fragrance precursor which is capable of releasing fragrance over an extended time period and providing a long-lasting release of said fragrance.

In accordance with a second aspect of the present invention there are provided compounds as precursors.

In accordance with a third aspect of the present invention there is provided a method to release a fragrance ketone or aldehyde.

In accordance with a fourth aspect of the present invention there are provided methods of making said perfume precursors, perfume preparations and consumer products.

In accordance with a fifth aspect of the present invention there are provided perfume preparations and consumer products comprising said compounds.

In accordance with a sixth aspect of the present invention there is provided the use of said perfume precursors and perfume preparations in consumer products, such as fabric care, personal care and household care products.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that nitrones can serve as fragrance precursors able to release fragrance compounds which possess a carbonyl group. These fragrance precursors provide a delayed release of the carbonyl compound over a longer period of time than by the use of the fragrance itself.

There is therefore provided herein a use of a compound of formula (I)

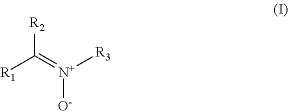

as precursor for generating a ketone or aldehyde of formula (II)

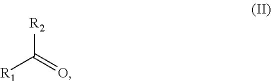

wherein
R₁ is selected from the group consisting of
linear or branched C₁-C₁₅ alkyl, for example C₅-C₁₄ such as C₆, C₇, C₈, C₉, C₁₀, C₁₁, C₁₂ or C₁₃ alkyl;
linear or branched C₁-C₁₅ hydroxyalkyl, alkoxyalkyl and alkylthioalkyl, for example C₅-C₁₄ such as C₆, C₇, C₈, C₉, C₁₀, C₁₁, C₁₂ or C₁₃ hydroxyalkyl, 4-(hexyloxy)butyl, 3-(methylthio)propyl;
linear or branched C₂-C₁₅alkenyl, for example C₅-C₁₄ such as C₆, C₇, C₈, C₉, C₁₀, C₁₁, C₁₂ or C₁₃ alkenyl comprising, e.g. one, two or three carbon-carbon double bonds;
linear or branched C₂-C₁₅ hydroxyalkenyl, for example C₅-C₁₄ such as C₆, C₇, C₈, C₉, C₁₀, C₁₁, C₁₂ or C₁₃ hydroxyalkenyl comprising, e.g. one, two or three carbon-carbon double bonds;
C₃-C₈ cycloalkyl such as cyclopentyl or cyclohexyl;
C₃-C₈ cycloalkyl (e.g. cyclopentyl or cyclohexyl) substituted with 1, 2, or 3 groups selected from C₁-C₇ alkyl (e.g. ethyl, isopropyl, tert-pentyl), C₂-C₇ alkenyl (e.g. isopropenyl), C₁-C₇ hydroxyalkyl, C₂-C₇ hydroxyalkenyl, and C₃-C₅ cycloalkyl;
C₅-C₈ cycloalkenyl (e.g. cyclopent-3-enyl, cyclohex-1-enyl, cyclohexa-2,4-dienyl, cyclooct-3-enyl);
C₅-C₈ cycloalkenyl (e.g. cyclohex-1-enyl, cyclohexa-2,4-dienyl, cyclooct-3-enyl) substituted with 1, 2, or 3 groups selected from C₁-C₇ alkyl (e.g. ethyl, isopropyl, tert-pentyl), C₂-C₇ alkenyl (e.g. isopropenyl), C₁-C₇ hydroxyalkyl, C₂-C₇ hydroxyalkenyl, and C₃-C₅ cycloalkyl (e.g. R₁ is 7-methyl-spiro[4.5]dec-8-en-6-yl, spiro[4.5]dec-7-en-7-yl, 5,5-dimethylcyclohex-1-enyl, 2,6,6-trimethylcyclohex-1,3-dienyl, 2,4-dimethylcyclohex-3-enyl, or 4-isopropenyl(cyclohex-1en-1yl);
(C₁-C₃)alkyl(C₅-C₆)cycloalkyl, wherein the cycloalkyl-ring is optionally substituted with one group selected from —OH group and ═O group, and/or one or two ether group(s), and/or up to four C₁-C₇ alkyl groups (e.g. R₁ is (3-tertbutylcyclohexyl)ethyl, or (4-(1,1-dimethylpropyl)cyclohexyl)methyl, (3-oxo-2-pentylcyclopentyl)methyl), C₂-C₇ alkenyl (e.g. isopropenyl), C₁-C₇ hydroxyalkyl, C₂-C₇ hydroxyalkenyl, and C₃-C₅ cycloalkyl;
(C₁-C₄)alkyl(C₅-C₆)cycloalkenyl wherein the cycloalkenyl-ring is optionally substituted with one —OH group, and/or one or two ether group(s), and/or up to four (i.e. 0, 1, 2, 3 or 4) C₁-C₇ alkyl groups (e.g. (2,6,6-trimethylcyclohex-1-en-1-yl)ethyl, 1-(2,6,6-trimethylcyclohex-2-en-1-yl)butan-3-yl), C₂-C₇ alkenyl (e.g. isopropenyl), C₁-C₇ hydroxyalkyl, C₂-C₇ hydroxyalkenyl, and C₃-C₅ cycloalkyl;
(C₂-C₃)alkenyl(C₅-C₆)cycloalkenyl wherein the cycloalkenyl-ring is optionally substituted with one —OH group, and/or one or two ether group(s), and/or up to four (i.e. 0, 1, 2, 3, or 4) C₁-C₇ alkyl groups (e.g. (2,6,6-trimethylcyclohex-1-en-1-yl)ethenyl, (2,6,6-trimethylcyclohex-2-en-1-yl)ethenyl, (2,6,6-trimethylcyclohex-2-en-1-yl)prop-2-en-2-yl, (2,6,6-trimethylcyclohex-1-en-1-yl)prop-2-en-2-yl), C₂-C₇ alkenyl (e.g. isopropenyl), C₁-C₇ hydroxyalkyl, C₂-C₇ hydroxyalkenyl, and C₃-C₅ cycloalkyl; C₆-C₁₄ aryl, e.g. phenyl or naphthyl; C₆-C₁₄ aryl wherein the aryl ring is substituted with up to 3 (i.e. 0, 1, 2, or 3) groups selected from C₁-C₄ alkyl (e.g. ethyl, iso-propyl, tert-butyl), —O—CH₂—O—, and —OR¹¹ wherein R¹¹ is independently selected from hydrogen and C₁-C₄ alkyl (e.g. ethyl, iso-propyl, tert-butyl);
(C₁-C₄)alkyl(C₆-C₁₄)aryl, e.g. benzyl, 2-phenylethyl;
(C₁-C₄)alkyl(C₆-C₁₄)aryl such as benzyl or 2-phenylethyl wherein the aryl-ring is substituted with up to 2 groups (e.g. 1 group) selected from C₁-C₄ alkyl (e.g. ethyl, iso-propyl, tert-butyl), —O—CH₂—O—, and —OR¹² wherein R¹² is independently selected from hydrogen and C₁-C₄ alkyl (e.g. ethyl, iso-propyl, tert-butyl);
(C₂-C₈)alkenyl(C₆-C₁₄)aryl, e.g. 2-phenylethylene-1-yl, 1-phenylhept-1-en-2-yl;
(C₂-C₈)alkenyl(C₆-C₁₄)aryl wherein the aryl-ring is substituted with up to 2 groups (e.g. 1 group) selected from C₁-C₄ alkyl (e.g. ethyl, iso-propyl, tert-butyl), —O—CH₂—O—, and —OR¹³ wherein R¹³ is independently selected from hydrogen and C₁-C₄ alkyl (e.g. ethyl, iso-propyl, tert-butyl);
bi-, or tricyclic hydrocarbon ring comprising C₈-C₁₂ carbon atoms, optionally having one CC double bond, optionally substituted with up to 6 groups selected from C₁-C₄ alkyl (e.g. ethyl, iso-propyl, tert-butyl), —O—CH₂—O—, and —OR¹⁴ wherein R¹⁴ is independently selected from hydrogen and C₁-C₄ alkyl (e.g. ethyl, iso-propyl, tert-butyl);
R₂ is selected from the group consisting of hydrogen, C₁-C₅ alkyl, and C₂-C₅ alkenyl;
or
R₁ and R₂ form together with the carbon atom to which they are attached a 5 to 16 membered hydrocarbon ring, the ring may optionally be substituted with C₁-C₅ alkyl groups (e.g. methyl, 3-methylbutyl, pentyl), or alkylacetate groups, for example, R₁ and R₂ forming together with the carbon atom to which they are attached 3-(2-methoxy-2-oxoethyl)-2-pentylcyclopent-1-yliden, cyclopentadecyliden;
R₃ is selected from the group consisting of
linear or branched C₁-C₁₅ alkyl, for example C₅-C₁₄ such as methyl, ethyl, propyl, iso-propyl, 2-butyl, 2-methylpentyl, octyl, decyl, dodecyl, 2-methylundecyl,
linear or branched C₂-C₁₅ alkenyl, for example C₅-C₁₄ such as 3-methylbut-2-en-1-yl;
C₃-C₈ cycloalkyl, optionally substituted, for example cyclobutyl, cyclopentyl, cyclohexyl, 2-isopropyl-5-methylcyclohexyl, cycloheptyl, cyclooctyl,
bi- or tricyclic hydrocarbon ring comprising C₈-C₁₂ carbon atoms, e.g. (1R,5S)-6,6-dimethylbicyclo[3.1.1]hept-2-en-2-yl)methyl (myrtenyl),
and aromatic residues selected from p-isopropylbenzyl, benzyl, p-methylbenzyl, p-methoxybenzyl, 1-phenylethyl, benzo[d][1,3]dioxol-5-ylmethyl, naphthalen-2-ylmethyl; 1-(naphthalen-2-yl)ethyl; phenyl, p-nitrobenzyl; 3-ethoxy-4-hydroxybenzylpyridin-2-ylmethyl and furan-2-ylmethyl.

The configuration of the CN double bond in the compound of formula (I) is not specified, so it has either E- or Z-configuration, or the compound is a mixture of E and Z isomers.

In R₁, R₂ and/or R₃, each double bond can have either E- or Z-configuration, or being an E-/Z-mixture, if not further specified.

Beside the ketone or aldehyde of formula (II) the compound of formula (I) further releases a nitrogen containing compound, for example an oxime (compound of formula (III)) or a nitrile (compound of formula (IV)), or fragments thereof.

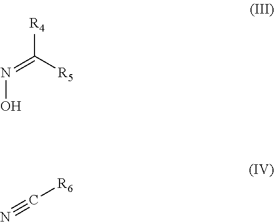

In the compound of formula (III), R₄ and R₅ form together with the carbon atom they are attached to the residue R₃ as defined for the compound of formula (I) above.

In the compound of formula (IV), R₆ forms together with the carbon atom it is attached to the residue R₃ as defined for the compound of formula (I) above.

Some subgroups of nitrones are reported in literature. For example, according to EP0311568, long chain N-alkyl-α-alkyl nitrones can be used as process stabilizers for organic materials. In US2004198926A1, the use of nitrones of Vanilin as curing agent for phenolic resins is described. However, the use of nitrones as fragrance precursors has not been disclosed.

According to one aspect of the invention, there is provided the use of the compound of formula (I) as precursor, with R₁ and R₂ is as defined above, and wherein R₃ is selected from the group consisting of isopropyl, cyclohexyl, benzyl and p-methylbenzyl.

For example, there is provided the use of the compound of formula (I) as precursor selected from the group consisting of (E)-N-isopropyl-2-methylundecan-1-imine oxide, 2-methyl-N-(4-methylbenzyl)undecan-1-imine oxide, N-cyclohexyl-2-methylundecan-1-imine oxide, N-benzylnona-2,6-dien-1-imine oxide, (2E,3E)-N-isopropyl-4-(2,6,6-trimethylcyclohex-1-en-1-yl)but-3-en-2-imine oxide, 2-methyl-N-(3-methylbut-2-en-1-yl)undecan-1-imine oxide, N-cyclobutyl-2-methylundecan-1-imine oxide, N-(furan-2-ylmethyl)-2-methylundecan-1-imine oxide, N,2-dimethylundecan-1-imine oxide, N-benzyl-2-methylundecan-1-imine oxide, N-ethyl-2-methylundecan-1-imine oxide, 2-methyl-N-(2-methylpentyl)undecan-1-imine oxide, N-(benzo[d][1,3]dioxol-5-ylmethyl)-2-methylundecan-1-imine oxide, N-(3-ethoxy-4-hydroxybenzyl)-2-methylundecan-1-imine oxide, 2-methyl-N-(naphthalen-2-ylmethyl)undecan-1-imine oxide, 2-methyl-N-(2-methylundecyl)undecan-1-imine oxide, N-(4-methoxybenzyl)-2-methylundecan-1-imine oxide, N-(sec-butyl)-2-methylundecan-1-imine oxide, 2-methyl-N-phenylundecan-1-imine oxide, N-cyclopentyl-2-methylundecan-1-imine oxide, 2-methyl-N-(4-nitrobenzyl)undecan-1-imine oxide, N-(cyclohexylmethyl)-2-methylundecan-1-imine oxide, N-(4-isopropylbenzyl)-2-methylundecan-1-imine oxide, N-(((1R,5S)-6,6-dimethylbicyclo[3.1.1]hept-2-en-2-yl)methyl)-2-methylundecan-1-imine oxide, N-(2-isopropyl-5-methylcyclohexyl)-2-methylundecan-1-imine oxide, 2-methyl-N-(1-phenylethyl)undecan-1-imine oxide, N-(sec-butyl)-2-methylundecan-1-imine oxide, 2-methyl-N-octylundecan-1-imine oxide, N-cycloheptyl-2-methylundecan-1-imine oxide, 2-methyl-N-(pyridin-2-ylmethyl)undecan-1-imine oxide, N-cyclooctyl-2-methylundecan-1-imine oxide, 2-methyl-N-(1-(naphthalen-2-yl)ethyl)undecan-1-imine oxide, 3-(4-isobutyl-2-methylphenyl)-N-methylpropan-1-imine oxide, N-benzyl-3-(4-isobutyl-2-methylphenyl)propan-1-imine oxide, N-ethyl-3-(4-isobutyl-2-methylphenyl)propan-1-imine oxide, 3-(4-isobutyl-2-methylphenyl)-N-(4-methoxybenzyl)propan-1-imine oxide, 3-(4-isobutyl-2-methylphenyl)-N-isopropylpropan-1-imine oxide, N-cyclohexyl-3-(4-isobutyl-2-methylphenyl)propan-1-imine oxide, N-(benzo[d][1,3]dioxol-5-ylmethyl)-3-(4-isobutyl-2-methylphenyl)propan-1-imine oxide, 3-(4-isobutyl-2-methylphenyl)-N-(1-(naphthalen-2-yl)ethyl)propan-1-imine oxide, 3-(4-isobutyl-2-methylphenyl)-N-(2-methylundecyl)propan-1-imine oxide, N-(sec-butyl)-3-(4-isobutyl-2-methylphenyl)propan-1-imine oxide, 3-(4-isobutyl-2-methylphenyl)-N-(1-phenylethyl)propan-1-imine oxide, N-cyclobutyl-3-(4-isobutyl-2-methylphenyl)propan-1-imine oxide, 3-(4-isobutyl-2-methylphenyl)-N-(3-methylbut-2-en-1-yl)propan-1-imine oxide, 1-(3-ethoxy-4-hydroxyphenyl)-N-methylmethanimine oxide, N-benzyl-1-(3-ethoxy-4-hydroxyphenyl)methanimine oxide, 1-(3-ethoxy-4-hydroxyphenyl)-N-ethylmethanimine oxide, 1-(3-ethoxy-4-hydroxyphenyl)-N-(4-methoxybenzyl)methanimine oxide, 1-(3-ethoxy-4-hydroxyphenyl)-N-isopropylmethanimine oxide, N-cyclohexyl-1-(3-ethoxy-4-hydroxyphenyl)methanimine oxide, N-(benzo[d][1,3]dioxol-5-ylmethyl)-1-(3-ethoxy-4-hydroxyphenyl)methanimine oxide, 1-(3-ethoxy-4-hydroxyphenyl)-N-(1-(naphthalen-2-yl)ethyl)methanimine oxide, 1-(3-ethoxy-4-hydroxyphenyl)-N-(2-methylundecyl)methanimine oxide, N-isopropyl-4-(4-methoxyphenyl)butan-2-imine oxide, N-isopropyldodecan-1-imine oxide, 7-hydroxy-N-isopropyl-3,7-dimethyloctan-1-imine oxide, 3-(4-isobutylphenyl)-N-isopropyl-2-methylpropan-1-imine oxide, N-isopropyl-2,6-dimethylhept-5-en-1-imine oxide, N-isopropyl-2-methyldecan-1-imine oxide, 1-(4-(4-hydroxy-4-methylpentyl)cyclohex-3-en-1-yl)-N-isopropylmethanimine oxide, 4-(4-hydroxyphenyl)-N-isopropylbutan-2-imine oxide, N-isopropyl-3,5,5-trimethylhexan-1-imine oxide, N-isopropyldec-9-en-1-imine oxide; N-isopropyl-3-(3-isopropylphenyl)butan-1-imine oxide, 3-(4-(tert-butyl)phenyl)-N-isopropylpropan-1-imine oxide, N-isopropyl-1-(naphthalen-2-yl)ethan-1-imine oxide, N-isopropyl-3-methylbutan-1-imine oxide, N-isopropyl-3,7-dimethyloct-6-en-1-imine oxide, N-isopropyl-2,4,7-trimethyloct-6-en-1-imine oxide, N-isopropyl-1-(1-methyl-2-(5-methylhex-4-en-2-yl)cyclopropyl)methanimine oxide, 1-(2,4-dimethylcyclohex-3-en-1-yl)-N-isopropylmethanimine oxide, N-isopropyl-3-(4-isopropylphenyl)-2-methylpropan-1-imine oxide, (2E)-N-isopropyl-3,7-dimethylocta-2,6-dien-1-imine oxide, (2E)-N-isopropylhex-2-en-1-imine oxide, (2E,6E)-N-isopropyl-3,7,11-trimethyldodeca-2,6,10-trien-1-imine oxide, N-isopropyl-1-(8-isopropyl-6-methylbicyclo[2.2.2]oct-5-en-2-yl)methanimine oxide; 1-(8,8-dimethyl-1,2,3,4,5,6,7,8-octahydronaphthalen-2-yl)-N-isopropylmethanimine oxide, N-isopropyl-1-(2,4,6-trimethylcyclohex-3-en-1-yl)methanimine oxide, N-isopropyl-4-(4-methoxyphenyl)butan-2-imine oxide, N-isopropyl-2-phenylethan-1-imine oxide, 9-hydroxy-N-isopropyl-5,9-dimethyldec-4-en-1-imine oxide, 1-(3-hydroxy-4-methoxyphenyl)-N-isopropylmethanimine oxide, N-isopropyl-1-(4-isopropylphenyl)methanimine oxide, (S)—N-isopropyl-1-(4-(prop-1-en-2-yl)cyclohex-1-en-1-yl)methanimine oxide, N-isopropyloctan-1-imine oxide, (3E)-N-isopropyl-4-(2,6,6-trimethylcyclohex-1-en-1-yl)but-3-en-2-imine oxide, N-isopropyl-3-(2-methoxy-2-oxoethyl)-2-pentylcyclopentan-1-imine oxide, (2E)-N-isopropyldodec-2-en-1-imine oxide, 3-(3-ethylphenyl)-N-isopropyl-2,2-dimethylpropan-1-imine oxide, N-isopropyl-3-(3-methoxyphenyl)-2-methylpropan-1-imine oxide, N-isopropyl-3-(methylthio)propan-1-imine oxide, 3-(4-(tert-butyl)phenyl)-N-isopropyl-2-methylpropan-1-imine oxide, N-isopropyl-7-methoxy-3,7-dimethyloctan-1-imine oxide, N-isopropyl-1-(3-methylbenzofuran-2-yl)ethan-1-imine oxide, 4-(hexyloxy)-N-isopropylbutan-1-imine oxide, (4Z)—N-isopropylhept-4-en-1-imine oxide, 3-(benzo[d][1,3]dioxol-5-yl)-N-isopropyl-2-methylpropan-1-imine oxide, (R)—N-isopropyl-2-(2,2,3-trimethylcyclopent-3-en-1-yl)ethan-1-imine oxide, 1-(4-(4-hydroxy-4-methylpentyl)cyclohex-3-en-1-yl)-N-methylmethanimine oxide, N-benzyl-1-(4-(4-hydroxy-4-methylpentyl)cyclohex-3-en-1-yl)methanimine oxide, 9-hydroxy-N,5,9-trimethyldec-4-en-1-imine oxide, N-benzyl-9-hydroxy-5,9-dimethyldec-4-en-1-imine oxide, (3E)-N-benzyl-4-(2,6,6-trimethylcyclohex-1-en-1-yl)but-3-en-2-imine oxide, (3Z)—N-benzylhex-3-en-1-imine oxide, N-benzyl-4-(hexyloxy)butan-1-imine oxide, N-benzyl-4-((6-methylheptan-2-yl)oxy)butan-1-imine oxide, (4Z)—N-benzylhept-4-en-1-imine oxide, N-benzyl-2-((3,7-dimethyloct-6-en-1-yl)oxy)ethan-1-imine oxide, (2E,6Z)—N-benzylnona-2,6-dien-1-imine oxide, N-(3-ethoxy-4-hydroxybenzyl)-1-(furan-2-yl)methanimine oxide, (2E)-N-benzyldodec-2-en-1-imine oxide, (2E)-N-benzyloct-2-en-1-imine oxide, (2E,6E)-N-benzyl-3,7-dimethylnona-2,6-dien-1-imine oxide, (2E)-N-benzyl-2-methyl-3-phenylprop-2-en-1-imine oxide, (2E)-N-benzyl-3,7-dimethylocta-2,6-dien-1-imine oxide, (2E)-N-benzylnon-2-en-1-imine oxide, 1-(4-(4-hydroxy-4-methylpentyl)cyclohex-3-en-1-yl)-N-(2-methylundecyl)methanimine oxide, 7-hydroxy-3,7-dimethyl-N-(2-methylundecyl)octan-1-imine oxide, (4E)-N-cyclohexyl-9-hydroxy-5,9-dimethyldec-4-en-1-imine oxide, N-cyclohexyl-1-(4-(4-hydroxy-4-methylpentyl)cyclohex-3-en-1-yl)methanimine oxide, N-benzyl-3-(3-isopropylphenyl)butan-1-imine oxide, N-benzyl-4-(4-hydroxyphenyl)butan-2-imine oxide, N-benzyl-2-methyldecan-1-imine oxide, N-benzyl-7-hydroxy-3,7-dimethyloctan-1-imine oxide, 3-(benzo[d][1,3]dioxol-5-yl)-N-benzyl-2-methylpropan-1-imine oxide, N-(2-methylundecyl)propan-1-imine oxide, N-benzyldec-9-en-1-imine oxide, 1-(4-hydroxy-3-methoxyphenyl)-N-isopropylmethanimine oxide, N-isopropyl-1-(4-methoxyphenyl)methanimine oxide, N-isopropylnonan-1-imine oxide, 1-(benzo[d][1,3]dioxol-5-yl)-N-isopropylmethanimine oxide, N-isopropyl-1-(p-tolyl)methanimine oxide, 1-(3,4-dimethoxyphenyl)-N-isopropylmethanimine oxide, N-isopropyl-2-methylpropan-1-imine oxide, N-isopropyldecan-1-imine oxide, N-isopropylhexan-1-imine oxide, 1-(furan-2-yl)-N-isopropylmethanimine oxide, N-benzyl-1-(4-methoxyphenyl)methanimine oxide, N-benzyldodecan-1-imine oxide and N-decyldecan-1-imine oxide.

The compounds of formula (I) described above release volatile compounds upon exposure to ambient air and/or humidity (by oxidation and/or hydrolysis) over a long period of time (e.g. several days such as 2-7 days or even longer). In one embodiment, at least one of the released compounds is a fragrant compound. For example, the compound of formula (II) is a fragrant aldehyde (for R₂═H) or ketone (for R₂≠H).

For example, the fragrant aldehyde (for R₂═H) or ketone (for R₂≠H) of formula (II) that can be released from the compound of formula (I) upon exposure to ambient air and/or humidity can be selected from the group consisting of 2-methylundecanal, 3-(4-isobutyl-2-methylphenyl)propanal, 3-ethoxy-4-hydroxybenzaldehyde, 4-(4-methoxyphenyl)butan-2-one, dodecanal, 4-hydroxy-3-methoxybenzaldehyde (Vanillin), 4-methoxybenzaldehyde (Anisaldehyde), 7-hydroxy-3,7-dimethyloctanal (hydroxycitronellal), 3-(4-isobutylphenyl)-2-methylpropanal (Silvial), 2,6-dimethylhept-5-enal (Melonal), 2-methyldecanal, 4-(4-hydroxy-4-methylpentyl)cyclohex-3-ene-1-carbaldehyde (Cyclohexal), 4-(4-hydroxyphenyl)butan-2-one (Raspberry Ketone), 3,5,5-trimethylhexanal (Isononylic aldehyde), nonanal, dec-9-enal, 3-(3-isopropylphenyl)butanal (Florhydral), 3-(4-(tert-butyl)phenyl)propanal (Burgeonal), 1-(naphthalen-2-yl)ethan-1-one (Oranger Crist), 3-methylbutanal, 3,7-dimethyloct-6-enal (Citronellal), 2,4,7-trimethyloct-6-enal (Pomelol), 1-methyl-2-(5-methylhex-4-en-2-yl)cyclopropane-1-carbaldehyde (Rosyfolia), 2,4-dimethylcyclohex-3-ene-1-carbaldehyde, 3-(4-isopropylphenyl)-2-methylpropanal (Cyclamen aldehyde), (E)-3,7-dimethylocta-2,6-dienal (Citral), benzo[d][1,3]dioxole-5-carbaldehyde (Heliotropine), hex-2-enal, (2E,6E)-3,7,11-trimethyldodeca-2,6,10-trienal (Farnesal), 8-isopropyl-6-methylbicyclo[2.2.2]oct-5-ene-2-carbaldehyde (Cyclomyral), 8,8-dimethyl-1,2,3,4,5,6,7,8-octahydronaphthalene-2-carbaldehyde (Maceal), 2,4,6-trimethylcyclohex-3-ene-1-carbaldehyde (Isocyclocitral), 4-methylbenzaldehyde, 3,4-dimethoxybenzaldehyde (Veratryl adlehyde), 4-(4-methoxyphenyl)butan-2-one, 2-phenylacetaldehyde, 9-hydroxy-5,9-dimethyldec-4-enal (Mahonial), 3-hydroxy-4-methoxybenzaldehyde, 4-isopropylbenzaldehyde (Cuminaldehyde), isobutyraldehyde, (S)-4-(prop-1-en-2-yl)cyclohex-1-ene-1-carbaldehyde (Perilla aldehyde), decanal, octanal, hexanal, (E)-4-(2,6,6-trimethylcyclohex-1-en-1-yl)but-3-en-2-one (β-Ionone), methyl 2-(3-oxo-2-pentylcyclopentyl)acetate (Hedione), (E)-dodec-2-enal (Mandarine aldehyde), 3-(3-ethylphenyl)-2,2-dimethylpropanal (Floralozone), 3-(3-methoxyphenyl)-2-methylpropanal, 3-(methylthio)propanal (Methional), 3-(4-(tert-butyl)phenyl)-2-methylpropanal (Lilial), 7-methoxy-3,7-dimethyloctanal, 3-methyl-2-(prop-1-en-2-yl)benzofuran, 4-(hexyloxy)butanal, (Z)-hept-4-enal, furan-2-carbaldehyde, 3-(benzo[d][1,3]dioxol-5-yl)-2-methylpropanal, (R)-2-(2,2,3-trimethylcyclopent-3-en-1-yl)acetaldehyde, (Z)-hex-3-enal, 4-((6-methylheptan-2-yl)oxy)butanal, (Z)-hept-4-enal, 2-((3,7-dimethyloct-6-en-1-yl)oxy)acetaldehyde, (2E,6Z)-nona-2,6-dienal, (E)-oct-2-enal, (2E,6E)-3,7-dimethylnona-2,6-dienal, (E)-2-methyl-3-phenylacrylaldehyde, (E)-3,7-dimethylocta-2,6-dienal, non-2-enal, 7-hydroxy-3,7-dimethyloctanal and 2-methyldecanal.

Furthermore, the fragrant aldehyde (for R₂═H) or ketone (for R₂≠H) of formula (II) that can be released from the compound of formula (I) upon exposure to ambient air and/or humidity can be selected from the group consisting of benzaldehyde, 2,6,10-trimethylundec-9-enal, 8,8-dimethyl-1,2,3,4,5,6,7,8-octahydro-naphthalene-2-carbaldehyde, (4-isopropyl-phenyl)-ethanal, 2,4-dimethyl-cyclohex-3-ene-1-carbaldehyde, 1,3,5-trimethyl-cyclohex-1-ene-4-carbaldehyde, 4-(4-hydroxy-4-methylpentyl)-cyclohex-3-ene-1-carbaldehyde, hex-2-enal, hex-3-enal, 3-(3-tert-butylcyclohexyl)propanal, 2-(4-tert-pentylcyclohexyl)acetaldehyde, 3,5,5-trimethyl-hexanal, heptanal, 2,6-dimethyl-hept-5-enal, dec-4-en-1-al, 2-methyl-decanal, undec-10-en-1-al, undecanal, tridecanal, tridec-2-enal, undec-9-enal, 2-phenyl-propanal, 2-(4-methyl-phenyl)-ethanal, 2-(4-methoxyphenyl)acetaldehyde, 3,7-dimethyl-octanal, dihydrofarnesal (3,7,11-trimethyldodeca-6,10-dienal), 7-hydroxy-3,7-dimethyl-octanal, 2,6-dimethyl-oct-5-en-1-al, 4-(4-methyl-pent-3-enyl)-cyclohex-3-ene-1-carbaldehyde, 2,3,5,5,-tetramethyl-hexanal, decahydro-4,8,8-trimethyl-1,4-methanoazulene-9-carboxaldehyde (longifolic aldehyde), 3-(4-tert-butyl-phenyl)-propanal, 3-(4-isobutyl-phenyl)-propanal, 3-(benzo[1,3]dioxol-5-yl)-2-methyl-propanal, 3,7-dimethyl-oct-6-ene-1-al, 2-methyl-3-(4-isopropylphenyl)-propanal, 4-tert-butyl-cyclohexane-1-carbaldehyde, 4-(octahydro-4,7-methano-5H-inden-5-ylidene)-butanal, (3,7-dimethyl-oct-6-enyloxy)-ethanal, 2 (E),6 (Z)-nonadienal, 2,4-dimethyl-2,6-heptadienal, (E)-dec-2-enal, dodec-2-enal, 3,7-dimethyl-octa-2,6-dienal, 2,4-diethyl-hepta-2,6-dienal, 3,7-dimethyl-nona-2,6-dienal, 2-propyl-hept-2-enal, 3-(4-methoxyphenyl)-2-methylpropanal, 1,3-benzodioxole-5-carboxaldehyde, 2,6,6-trimethylcyclohexa-1,3-dienecarbaldehyde, 4-((6-methylheptan-2-yl)oxy)butanal, 3-(4-ethylphenyl)-2,2-dimethylpropanal, (E)-2-benzylideneheptanal, cinnamaldehyde, 3-(3,3-dimethyl-2,3-dihydro-1H-inden-5-yl)propanal, 2-methyl-4-(2,6,6-trimethylcyclohex-2-en-1-yl)butanal (Cetonal), 4-isopropenyl-cyclohex-1-ene-1-carbaldehyde, further lonones such as alpha-lonone, dihydro alpha-Ionone, dihydro beta-Ionone, N-allyl alpha-ionone (1-(2,6,6-trimethyl-2-cyclohexen-1-yl)-1,6-heptadien-3-one), N-methyl alpha-ionone (1-(2,6,6-trimethylcyclohex-2-en-1-yl)pent-1-en-3-one), dimethyl alpha-ionone (2-methyl-1-(2,6,6-trimethylcyclohex-2-en-1-yl)pent-1-en-3-one), and Cetone Alpha (3-methyl-4-(2,6,6-trimethylcyclohex-2-en-1-yl)but-3-en-2-one), Nectaryl (2-(2-(4-methylcyclohex-3-en-1-yl)propyl)cyclopentanone), Magnolione (3-(2-oxopropyl)-2-pentylcyclopentanone), Acetophenone and Cassione (4-(benzo[d][1,3]dioxol-5-yl)butan-2-one).

For example, the fragrant aldehyde (for R²═H) or ketone (for R²≠H) of formula (II) that can be released from the compound of formula (I) upon on exposure to ambient air and/or humidity is a compound having at least 4 carbon atoms or more, for example at least 6 carbon atoms or more.

Exposure of the precursor compound to ambient air means exposure to molecular oxygen which might be responsible for the cleavage of the compound of formula (I) and the release of the compound of formula (II). The concentration of oxygen in the air is sufficient for cleaving the compound of formula (I) so that the cleavage products can be detected in the ambient air, e.g. by olfaction or GC-MS analysis of headspace samples.

Exposure of the precursor compound to humidity means exposure to water which might be responsible for the cleavage of the compound of formula (I) and the release of the compound of formula (II). Already traces of water are sufficient to allow for cleavage of the compound of formula (I).

The compounds of formula (I) are very stable when not exposed to the ambient air or humidity, i.e. when stored in neat form in appropriate containers protected from air and humidity, or when stored in proper solvents, for example in dipropylene glycol, isopropyl myristate, triethyl citrate, or when incorporated in consumer products such as detergent, shampoo and fabric conditioner. Thus the compounds of formula (I) may find use in a broad range of consumer products in which a prolonged and defined release of fragrant compounds is desired. In some embodiments, stabilizing compounds, for example Tocopherol can be added to the compounds of formula (I), for example in 0.01-0.5% by weight.

For example, the compounds of formula (I) can act as fragrance precursors in consumer products, for example in fabric care, personal care and home care products or in fine fragrance. In particular, these precursors are well suited for fabric care applications.

Personal care products to which the compound of formula (I) can be added include for example all kinds of body care products. Especially interesting products are hair care products, for example shampoos, conditioners and hairsprays, and skin care products, like lotions or creams. Furthermore, the compounds of formula (I) may be added to soaps, bath and shower gels and deodorants.

Home cleaning products to which the compound of formula (I) can be added include all kinds of detergents, window cleaners, hard surface cleaners, all-purpose cleaners and furniture polishes. Preferably, the products are liquids, e.g. fabric conditioner compositions. A substrate, such as a fabric, treated with a product comprising a compound of formula (I), will diffuse a fresh and/or clean odor under cleavage conditions for much longer than one treated with a conventional product. Fabrics or clothes washed with such a fabric softener will exhibit noticeable fragrance release even after one week.

For example, the compounds of formula (I) can act as fragrance precursors in consumer products which further comprise enzymes.

The compounds of formula (I) may be employed in a consumer product base simply by directly mixing the compound of formula (I), or a fragrance composition comprising a compound of formula (I) with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article, comprising the incorporation of a compound of formula (I), as a fragrance ingredient, either by directly admixing it to the consumer product base or by admixing a fragrance composition comprising the compound of formula (I), which may then be mixed with a consumer product base, using conventional techniques and methods.

The invention also provides a fragranced article comprising:
a) the compound of formula (I), or a mixture comprising it as odorant; and
b) a consumer product base.

As used herein, ‘consumer product base’ means a composition for use as a consumer product to fulfil specific actions, such as cleaning, softening, and caring or the like.

The compounds of formula (I) can be used alone, as a mixture thereof, or in combination with other fragrance ingredients and/or precursors thereof. Such fragrance ingredients are described, for example, in “Perfume and Flavor Chemicals”, S. Arctander, Ed., Vol. I & II, Allured Publishing Corporation, Carol Stream, USA, 2003 and include fragrance compounds of natural or synthetic origin and essential oils.

The amounts in which the compounds of formula (I) may be incorporated in the various above-mentioned products vary within a wide range. The amounts depend on the nature of the fragrant compounds to be released, the nature of the product to which the compounds of formula (I) are added and the desired olfactory effect. The amounts used also depend on the co-ingredients in a given composition when the compounds of formula (I) are used in admixture with perfuming co-ingredients, solvents or adjuvants. Typical concentrations are from 0.0001 to 5 weight percent of the article. In one embodiment, compounds of the present invention may be employed in a fabric softener in an amount of from 0.0001 to 0.2 weight percent. In another embodiment, compounds of the present invention may be used in a liquid detergent in an amount from 0.001 to 0.5 weight percent (e.g. up to about 5 weight percent), more preferably between 0.02 and 4 weight percent. In another embodiment, compounds of the present invention may be used in a shampoo in an amount from 0.001 to 0.5 weight percent. However, these values are given only by way of example, since the experienced perfumer may also achieve effects or may create novel accords with lower or higher concentrations.

Some of the compounds according to formula (I) are known from different application, however, most of the compounds are novel.

Therefore, the invention provides a compound of formula (I)

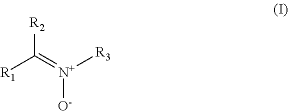

as precursor for generating a ketone or aldehyde of formula (II)

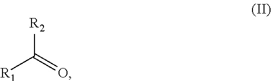

wherein
R₁ is selected from the group consisting of
linear or branched C₁-C₁₅ alkyl, for example C₅-C₁₄ such as C₆, C₇, C₈, C₉, C₁₀, C₁₁, C₁₂ or C₁₃ alkyl;
linear or branched C₁-C₁₅ hydroxyalkyl, alkoxyalkyl and alkylthioalkyl, for example C₅-C₁₄ such as C₆, C₇, C₈, C₉, C₁₀, C₁₁, C₁₂ or C₁₃ hydroxyalkyl, 4-(hexyloxy)butyl, 3-(methylthio)propyl;
linear or branched C₂-C₁₅alkenyl, for example C₅-C₁₄ such as C₆, C₇, C₈, C₉, C₁₀, C₁₁, C₁₂ or C₁₃ alkenyl comprising, e.g. one, two or three carbon-carbon double bonds;
linear or branched C₂-C₁₅ hydroxyalkenyl, for example C₅-C₁₄ such as C₆, C₇, C₈, C₉, C₁₀, C₁₁, C₁₂ or C₁₃ hydroxyalkenyl comprising, e.g. one, two or three carbon-carbon double bonds;
C₃-C₈ cycloalkyl such as cyclopentyl or cyclohexyl;
C₃-C₈ cycloalkyl (e.g. cyclopentyl or cyclohexyl) substituted with 1, 2, or 3 groups selected from C₁-C₇ alkyl (e.g. ethyl, isopropyl, tert-pentyl), C₂-C₇ alkenyl (e.g. isopropenyl), C₁-C₇ hydroxyalkyl, C₂-C₇ hydroxyalkenyl, and C₃-C₅ cycloalkyl;
C₅-C₈ cycloalkenyl (e.g. cyclopent-3-enyl, cyclohex-1-enyl, cyclohexa-2,4-dienyl, cyclooct-3-enyl);
C₅-C₈ cycloalkenyl (e.g. cyclohex-1-enyl, cyclohexa-2,4-dienyl, cyclooct-3-enyl) substituted with 1, 2, or 3 groups selected from C₁-C₇ alkyl (e.g. ethyl, isopropyl, tert-pentyl), C₂-C₇ alkenyl (e.g. isopropenyl), C₁-C₇ hydroxyalkyl, C₂-C₇ hydroxyalkenyl, and C₃-C₅ cycloalkyl (e.g. R₁ is 7-methyl-spiro[4.5]dec-8-en-6-yl, spiro[4.5]dec-7-en-7-yl, 5,5-dimethylcyclohex-1-enyl, 2,6,6-trimethylcyclohex-1,3-dienyl, 2,4-dimethylcyclohex-3-enyl, or 4-isopropenyl(cyclohex-1en-1yl);
(C₁-C₃)alkyl(C₅-C₆)cycloalkyl, wherein the cycloalkyl-ring is optionally substituted with one group selected from —OH group and ═O group, and/or one or two ether group(s), and/or up to four C₁-C₇ alkyl groups (e.g. R₁ is (3-tertbutylcyclohexyl)ethyl, or (4-(1,1-dimethylpropyl)cyclohexyl)methyl, (3-oxo-2-pentylcyclopentyl)methyl), C₂-C₇ alkenyl (e.g. isopropenyl), C₁-C₇ hydroxyalkyl, C₂-C₇ hydroxyalkenyl, and C₃-C₅ cycloalkyl;
(C₁-C₄)alkyl(C₅-C₆)cycloalkenyl wherein the cycloalkenyl-ring is optionally substituted with one —OH group, and/or one or two ether group(s), and/or up to four (i.e. 0, 1, 2, 3 or 4) C₁-C₇ alkyl groups (e.g. (2,6,6-trimethylcyclohex-1-en-1-yl)ethyl, 1-(2,6,6-trimethylcyclohex-2-en-1-yl)butan-3-yl), C₂-C₇ alkenyl (e.g. isopropenyl), C₁-C₇ hydroxyalkyl, C₂-C₇ hydroxyalkenyl, and C₃-C₅ cycloalkyl;
(C₂-C₃)alkenyl(C₅-C₆)cycloalkenyl wherein the cycloalkenyl-ring is optionally substituted with one —OH group, and/or one or two ether group(s), and/or up to four (i.e. 0, 1, 2, 3, or 4) C₁-C₇ alkyl groups (e.g. (2,6,6-trimethylcyclohex-1-en-1-yl)ethenyl, (2,6,6-trimethylcyclohex-2-en-1-yl)ethenyl, (2,6,6-trimethylcyclohex-2-en-1-yl)prop-2-en-2-yl, (2,6,6-trimethylcyclohex-1-en-1-yl)prop-2-en-2-yl), C₂-C₇ alkenyl (e.g. isopropenyl), C₁-C₇ hydroxyalkyl, C₂-C₇ hydroxyalkenyl, and C₃-C₅ cycloalkyl;
C₆-C₁₄ aryl, e.g. phenyl or naphthyl;
C₆-C₁₄ aryl wherein the aryl ring is substituted with up to 3 (i.e. 0, 1, 2, or 3) groups selected from C₁-C₄ alkyl (e.g. ethyl, iso-propyl, tert-butyl), —O—CH₂—O—, and —OR¹¹ wherein R¹¹ is independently selected from hydrogen and C₁-C₄ alkyl (e.g. ethyl, iso-propyl, tert-butyl);
(C₁-C₄)alkyl(C₆-C₁₄)aryl, e.g. benzyl, 2-phenylethyl;
(C₁-C₄)alkyl(C₆-C₁₄)aryl such as benzyl or 2-phenylethyl wherein the aryl-ring is substituted with up to 2 groups (e.g. 1 group) selected from C₁-C₄ alkyl (e.g. ethyl, iso-propyl, tert-butyl), —O—CH₂—O—, and —OR¹² wherein R¹² is independently selected from hydrogen and C₁-C₄ alkyl (e.g. ethyl, iso-propyl, tert-butyl);
(C₂-C₈)alkenyl(C₆-C₁₄)aryl, e.g. 2-phenylethylene-1-yl, 1-phenylhept-1-en-2-yl;
(C₂-C₈)alkenyl(C₆-C₁₄)aryl wherein the aryl-ring is substituted with up to 2 groups (e.g. 1 group) selected from C₁-C₄ alkyl (e.g. ethyl, iso-propyl, tert-butyl), —O—CH₂—O—, and —OR¹³ wherein R¹³ is independently selected from hydrogen and C₁-C₄ alkyl (e.g. ethyl, iso-propyl, tert-butyl);
bi- or tricyclic hydrocarbon ring comprising C₈-C₁₂ carbon atoms, optionally having one CC double bond, optionally substituted with up to 6 groups selected from C₁-C₄ alkyl (e.g. ethyl, iso-propyl, tert-butyl), —O—CH₂—O—, and —OR¹⁴ wherein R¹⁴ is independently selected from hydrogen and C₁-C₄ alkyl (e.g. ethyl, iso-propyl, tert-butyl); R₂ is selected from the group consisting of hydrogen, C₁-C₅ alkyl, and C₂-C₅ alkenyl;
or
R₁ and R₂ form together with the carbon atom to which they are attached a 5 to 16 membered hydrocarbon ring, the ring may optionally be substituted with C₁-C₅ alkyl groups (e.g. methyl, 3-methylbutyl, pentyl), or alkylacetate groups, for example, R₁ and R₂ forming together with the carbon atom to which they are attached 3-(2-methoxy-2-oxoethyl)-2-pentylcyclopent-1-yliden, cyclopentadecyliden;
R₃ is selected from the group consisting of
linear or branched C₁-C₁₅ alkyl, for example C₅-C₁₄ such as methyl, ethyl, propyl, iso-propyl, 2-butyl, 2-methylpentyl, octyl, decyl, dodecyl, 2-methylundecyl, linear or branched C₂-C₁₅ alkenyl, for example C₅-C₁₄ such as 3-methylbut-2-en-1-yl;
C₃-C₈ cycloalkyl, optionally substituted, for example cyclobutyl, cyclopentyl, cyclohexyl, 2-isopropyl-5-methylcyclohexyl, cycloheptyl, cyclooctyl,
bi-, or tricyclic hydrocarbon ring comprising C₈-C₁₂ carbon atoms, e.g. (1R,5S)-6,6-dimethylbicyclo[3.1.1]hept-2-en-2-yl)methyl (myrtenyl),
and aromatic residues selected from p-isopropylbenzyl, benzyl, p-methylbenzyl, p-methoxybenzyl, 1-phenylethyl, benzo[d][1,3]dioxol-5-ylmethyl, naphthalen-2-ylmethyl; 1-(naphthalen-2-yl)ethyl; phenyl, p-nitrobenzyl; 3-ethoxy-4-hydroxybenzylpyridin-2-ylmethyl and furan-2-ylmethyl;
with the proviso, that the compound is not 1-(4-hydroxy-3-methoxyphenyl)-N-isopropylmethanimine oxide, N-isopropyl-1-(4-methoxyphenyl)methanimine oxide, N-isopropylnonan-1-imine oxide, 1-(benzo[d][1,3]dioxol-5-yl)-N-isopropylmethanimine oxide, N-isopropyl-1-(p-tolyl)methanimine oxide, 1-(3,4-dimethoxyphenyl)-N-isopropylmethanimine oxide, N-isopropyl-2-methylpropan-1-imine oxide, N-isopropyldecan-1-imine oxide, N-isopropylhexan-1-imine oxide, 1-(furan-2-yl)-N-isopropylmethanimine oxide, N-benzyl-1-(4-methoxyphenyl)methanimine oxide, N-benzyldodecan-1-imine oxide, N-decyldecan-1-imine oxide.

The configuration of the CN double bond in the compound of formula (I) is not specified, so it has either E- or Z-configuration, or the compound is a mixture of E and Z isomers.

In R₁, R₂ and/or R₃, each double bond can have either E- or Z-configuration, or being an E-/Z-mixture, if not further specified.

For example, the compound of formula (I) is selected from the group consisting of (E)-N-isopropyl-2-methylundecan-1-imine oxide, 2-methyl-N-(4-methylbenzyl)undecan-1-imine oxide, N-cyclohexyl-2-methylundecan-1-imine oxide, N-benzylnona-2,6-dien-1-imine oxide, (2E,3E)-N-isopropyl-4-(2,6,6-trimethylcyclohex-1-en-1-yl)but-3-en-2-imine oxide, 2-methyl-N-(3-methylbut-2-en-1-yl)undecan-1-imine oxide, N-cyclobutyl-2-methylundecan-1-imine oxide, N-(furan-2-ylmethyl)-2-methylundecan-1-imine oxide, N,2-dimethylundecan-1-imine oxide, N-benzyl-2-methylundecan-1-imine oxide, N-ethyl-2-methylundecan-1-imine oxide, 2-methyl-N-(2-methylpentyl)undecan-1-imine oxide, N-(benzo[d][1,3]dioxol-5-ylmethyl)-2-methylundecan-1-imine oxide, N-(3-ethoxy-4-hydroxybenzyl)-2-methylundecan-1-imine oxide, 2-methyl-N-(naphthalen-2-ylmethyl)undecan-1-imine oxide, 2-methyl-N-(2-methylundecyl)undecan-1-imine oxide, N-(sec-butyl)-2-methylundecan-1-imine oxide, 2-methyl-N-phenylundecan-1-imine oxide, N-cyclopentyl-2-methylundecan-1-imine oxide, 2-methyl-N-(4-nitrobenzyl)undecan-1-imine oxide, N-(cyclohexylmethyl)-2-methylundecan-1-imine oxide, N-(4-isopropylbenzyl)-2-methylundecan-1-imine oxide, N-(((1R,5S)-6,6-dimethylbicyclo[3.1.1]hept-2-en-2-yl)methyl)-2-methylundecan-1-imine oxide, N-(2-isopropyl-5-methylcyclohexyl)-2-methylundecan-1-imine oxide, 2-methyl-N-(1-phenylethyl)undecan-1-imine oxide, N-(sec-butyl)-2-methylundecan-1-imine oxide, 2-methyl-N-octylundecan-1-imine oxide, N-cycloheptyl-2-methylundecan-1-imine oxide, 2-methyl-N-(pyridin-2-ylmethyl)undecan-1-imine oxide, N-cyclooctyl-2-methylundecan-1-imine oxide, 2-methyl-N-(1-(naphthalen-2-yl)ethyl)undecan-1-imine oxide, 3-(4-isobutyl-2-methylphenyl)-N-methylpropan-1-imine oxide, N-benzyl-3-(4-isobutyl-2-methylphenyl)propan-1-imine oxide, N-ethyl-3-(4-isobutyl-2-methylphenyl)propan-1-imine oxide, 3-(4-isobutyl-2-methylphenyl)-N-(4-methoxybenzyl)propan-1-imine oxide, 3-(4-isobutyl-2-methylphenyl)-N-isopropylpropan-1-imine oxide, N-cyclohexyl-3-(4-isobutyl-2-methylphenyl)propan-1-imine oxide, N-(benzo[d][1,3]dioxol-5-ylmethyl)-3-(4-isobutyl-2-methylphenyl)propan-1-imine oxide, 3-(4-isobutyl-2-methylphenyl)-N-(1-(naphthalen-2-yl)ethyl)propan-1-imine oxide, 3-(4-isobutyl-2-methylphenyl)-N-(2-methylundecyl)propan-1-imine oxide, N-(sec-butyl)-3-(4-isobutyl-2-methylphenyl)propan-1-imine oxide, 3-(4-isobutyl-2-methylphenyl)-N-(1-phenylethyl)propan-1-imine oxide, N-cyclobutyl-3-(4-isobutyl-2-methylphenyl)propan-1-imine oxide, 3-(4-isobutyl-2-methylphenyl)-N-(3-methylbut-2-en-1-yl)propan-1-imine oxide, 1-(3-ethoxy-4-hydroxyphenyl)-N-methylmethanimine oxide, N-benzyl-1-(3-ethoxy-4-hydroxyphenyl)methanimine oxide, 1-(3-ethoxy-4-hydroxyphenyl)-N-ethylmethanimine oxide, 1-(3-ethoxy-4-hydroxyphenyl)-N-(4-methoxybenzyl)methanimine oxide, 1-(3-ethoxy-4-hydroxyphenyl)-N-isopropylmethanimine oxide, N-cyclohexyl-1-(3-ethoxy-4-hydroxyphenyl)methanimine oxide, N-(benzo[d][1,3]dioxol-5-ylmethyl)-1-(3-ethoxy-4-hydroxyphenyl)methanimine oxide, 1-(3-ethoxy-4-hydroxyphenyl)-N-(1-(naphthalen-2-yl)ethyl)methanimine oxide, 1-(3-ethoxy-4-hydroxyphenyl)-N-(2-methylundecyl)methanimine oxide, N-isopropyl-4-(4-methoxyphenyl)butan-2-imine oxide, N-isopropyldodecan-1-imine oxide, 7-hydroxy-N-isopropyl-3,7-dimethyloctan-1-imine oxide, 3-(4-isobutylphenyl)-N-isopropyl-2-methylpropan-1-imine oxide, N-isopropyl-2,6-dimethylhept-5-en-1-imine oxide, N-isopropyl-2-methyldecan-1-imine oxide, 1-(4-(4-hydroxy-4-methylpentyl)cyclohex-3-en-1-yl)-N-isopropylmethanimine oxide, 4-(4-hydroxyphenyl)-N-isopropylbutan-2-imine oxide, N-isopropyl-3,5,5-trimethylhexan-1-imine oxide, N-isopropyldec-9-en-1-imine oxide; N-isopropyl-3-(3-isopropylphenyl)butan-1-imine oxide, 3-(4-(tert-butyl)phenyl)-N-isopropylpropan-1-imine oxide, N-isopropyl-1-(naphthalen-2-yl)ethan-1-imine oxide, N-isopropyl-3-methylbutan-1-imine oxide, N-isopropyl-3,7-dimethyloct-6-en-1-imine oxide, N-isopropyl-2,4,7-trimethyloct-6-en-1-imine oxide, N-isopropyl-1-(1-methyl-2-(5-methylhex-4-en-2-yl)cyclopropyl)methanimine oxide, 1-(2,4-dimethylcyclohex-3-en-1-yl)-N-isopropylmethanimine oxide, N-isopropyl-3-(4-isopropylphenyl)-2-methylpropan-1-imine oxide, (2E)-N-isopropyl-3,7-dimethylocta-2,6-dien-1-imine oxide, (2E)-N-isopropylhex-2-en-1-imine oxide, (2E,6E)-N-isopropyl-3,7,11-trimethyldodeca-2,6,10-trien-1-imine oxide, N-isopropyl-1-(8-isopropyl-6-methylbicyclo[2.2.2]oct-5-en-2-yl)methanimine oxide; 1-(8,8-dimethyl-1,2,3,4,5,6,7,8-octahydronaphthalen-2-yl)-N-isopropylmethanimine oxide, N-isopropyl-1-(2,4,6-trimethylcyclohex-3-en-1-yl)methanimine oxide, N-isopropyl-4-(4-methoxyphenyl)butan-2-imine oxide, N-isopropyl-2-phenylethan-1-imine oxide, 9-hydroxy-N-isopropyl-5,9-dimethyldec-4-en-1-imine oxide, 1-(3-hydroxy-4-methoxyphenyl)-N-isopropylmethanimine oxide, N-isopropyl-1-(4-isopropylphenyl)methanimine oxide, (S)—N-isopropyl-1-(4-(prop-1-en-2-yl)cyclohex-1-en-1-yl)methanimine oxide, N-isopropyloctan-1-imine oxide, (3E)-N-isopropyl-4-(2,6,6-trimethylcyclohex-1-en-1-yl)but-3-en-2-imine oxide, N-isopropyl-3-(2-methoxy-2-oxoethyl)-2-pentylcyclopentan-1-imine oxide, (2E)-N-isopropyldodec-2-en-1-imine oxide, 3-(3-ethylphenyl)-N-isopropyl-2,2-dimethylpropan-1-imine oxide, N-isopropyl-3-(3-methoxyphenyl)-2-methylpropan-1-imine oxide, N-isopropyl-3-(methylthio)propan-1-imine oxide, 3-(4-(tert-butyl)phenyl)-N-isopropyl-2-methylpropan-1-imine oxide, N-isopropyl-7-methoxy-3,7-dimethyloctan-1-imine oxide, N-isopropyl-1-(3-methylbenzofuran-2-yl)ethan-1-imine oxide, 4-(hexyloxy)-N-isopropylbutan-1-imine oxide, (4Z)—N-isopropylhept-4-en-1-imine oxide, 3-(benzo[d][1,3]dioxol-5-yl)-N-isopropyl-2-methylpropan-1-imine oxide, (R)—N-isopropyl-2-(2,2,3-trimethylcyclopent-3-en-1-yl)ethan-1-imine oxide, 1-(4-(4-hydroxy-4-methylpentyl)cyclohex-3-en-1-yl)-N-methylmethanimine oxide, N-benzyl-1-(4-(4-hydroxy-4-methylpentyl)cyclohex-3-en-1-yl)methanimine oxide, 9-hydroxy-N,5,9-trimethyldec-4-en-1-imine oxide, N-benzyl-9-hydroxy-5,9-dimethyldec-4-en-1-imine oxide, (3E)-N-benzyl-4-(2,6,6-trimethylcyclohex-1-en-1-yl)but-3-en-2-imine oxide, (3Z)—N-benzylhex-3-en-1-imine oxide, N-benzyl-4-(hexyloxy)butan-1-imine oxide, N-benzyl-4-((6-methylheptan-2-yl)oxy)butan-1-imine oxide, (4Z)—N-benzylhept-4-en-1-imine oxide, N-benzyl-2-((3,7-dimethyloct-6-en-1-yl)oxy)ethan-1-imine oxide, (2E,6Z)—N-benzylnona-2,6-dien-1-imine oxide, N-(3-ethoxy-4-hydroxybenzyl)-1-(furan-2-yl)methanimine oxide, (2E)-N-benzyldodec-2-en-1-imine oxide, (2E)-N-benzyloct-2-en-1-imine oxide, (2E,6E)-N-benzyl-3,7-dimethylnona-2,6-dien-1-imine oxide, (2E)-N-benzyl-2-methyl-3-phenylprop-2-en-1-imine oxide, (2E)-N-benzyl-3,7-dimethylocta-2,6-dien-1-imine oxide, (2E)-N-benzylnon-2-en-1-imine oxide, 1-(4-(4-hydroxy-4-methylpentyl)cyclohex-3-en-1-yl)-N-(2-methylundecyl)methanimine oxide, 7-hydroxy-3,7-dimethyl-N-(2-methylundecyl)octan-1-imine oxide, (4E)-N-cyclohexyl-9-hydroxy-5,9-dimethyldec-4-en-1-imine oxide, N-cyclohexyl-1-(4-(4-hydroxy-4-methylpentyl)cyclohex-3-en-1-yl)methanimine oxide, N-benzyl-3-(3-isopropylphenyl)butan-1-imine oxide, N-benzyl-4-(4-hydroxyphenyl)butan-2-imine oxide, N-benzyl-2-methyldecan-1-imine oxide, N-benzyl-7-hydroxy-3,7-dimethyloctan-1-imine oxide, 3-(benzo[d][1,3]dioxol-5-yl)-N-benzyl-2-methylpropan-1-imine oxide, N-(2-methylundecyl)propan-1-imine oxide and N-benzyldec-9-en-1-imine oxide.

The compounds of formula (I) may be prepared starting from the respective fragrant carbonyl compound of formula (II) as hereinabove defined and a hydroxylamine (compound of formula (V)). For example, an oxime (compound of formula (III)) is converted in a first step into the corresponding hydroxylamine hydrochloride, which is then reacted with a carbonyl compound (compound of formula (II)) to yield the nitrone (compound of formula (I)).

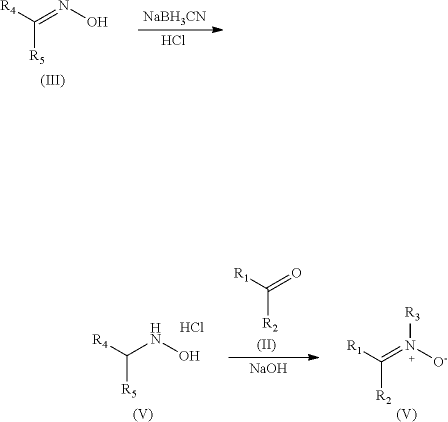

In the compound of formula (III), R₄ and R₅ form together with the carbon atom they are attached to the residue R₃ as defined for the compound of formula (I) above.

For example, the hydroxylamine of formula (V) that forms together with the compound of formula (II) the compound of formula (I) can be selected from the group consisting of N-isopropylhydroxylamine, N-(4-methylbenzyl)hydroxylamine, N-cyclohexylhydroxylamine, N-(3-methylbut-2-en-1-yl)hydroxylamine, N-cyclobutylhydroxylamine, N-(furan-2-ylmethyl)hydroxylamine, N-methylhydroxylamine, N-benzylhydroxylamine, N-ethylhydroxylamine, N-(2-methylpentyl)hydroxylamine, N-(benzo[d][1,3]dioxol-5-ylmethyl)hydroxylamine, N-(3-ethoxy-4-hydroxybenzyl)hydroxylamine, N-(naphthalen-2-ylmethyl)hydroxylamine, N-(2-methylundecyl)hydroxylamine, N-(sec-butyl)hydroxylamine, N-phenylhydroxylamine, N-cyclopentylhydroxylamine, N-(4-nitrobenzyl)hydroxylamine, N-(cyclohexylmethyl)hydroxylamine, N-(4-isopropylbenzyl)hydroxylamine, N-(((1R,5S)-6,6-dimethylbicyclo[3.1.1]hept-2-en-2-yl)methyl)hydroxylamine, N-(2-isopropyl-5-methylcyclohexyl)hydroxylamine, N-(1-phenylethyl)hydroxylamine, N-(sec-butyl)hydroxylamine, N-octylhydroxylamine, N-cycloheptylhydroxylamine, N-(pyridin-2-ylmethyl)hydroxylamine, N-cyclooctylhydroxylamine, N-(1-(naphthalen-2-yl)ethyl)hydroxylamine, N-(4-methoxybenzyl)hydroxylamine.

In some embodiments, the hydroxylamine hydrochloride can be obtained from an amine via an oxidative method. For example, a dialkylamine can be oxidized in the presence of a catalyst with H₂O₂ to a methanimine oxide, which is further converted to the desired hydroxylamine hydrochloride.

Alternatively, the compounds of formula (I) may be prepared directly in one step from the hydroxylamine and the aldehyde, if the reactivity of the hydroxylamine is sufficient. For example, N-isopropylhydroxylamine can be reacted directly with the carbonyl compound.

The compounds of formula (I) are obtained as solids or in liquid form.

In a further aspect, the compounds of the present invention are biodegradable, as demonstrated by the Manometric Respirometry test (OECD guideline for the testing of materials No. 301F, Paris 1992).

The invention is now further described with reference to the following non-limiting examples. These examples are for the purpose of illustration only and it is understood that variations and modifications can be made by one skilled in the art.

### DESCRIPTION OF FIGURES

FIG. 1 shows the comparison of a precursor and a free aldehyde used in a liquid detergent.

FIG. 2 shows the stability of a precursor in a liquid detergent.

### EXAMPLES

### General:

All reactions were performed under argon using solvents and reagents from commercial suppliers without further purification. Solvents for extraction and chromatography were technical grade and used without further purification. Flash chromatography was performed using Tsingdao Haiyang Chemical silica gel (200-300 mesh) and Santai Technologies silica flash columns. Unless otherwise noted, a mixture of Heptane: MTBE was used as eluent. NMR spectra were recorded with AW 400 MHz Bruker spectrometer instrument. The chemical shifts for ¹H NMR spectra was reported in b (ppm) referenced to the residual proton signal of the deuterated solvent; coupling constants were expressed in Hertz (Hz). ¹³C NMR spectra were referenced to the carbon signals of the deuterated solvent. The following abbreviations are used: s=singlet, d=doublet, t=triplet, q=quartet, m=multiplet, dd=double doublet, bs=broad singlet. GC/MS spectral data were obtained from an Agilent 6890 N and MSD 5975 using a column HP-5 MS, 30 m, 0.25 mm, 0.25 μm. High resolution mass spectra were determined on a Thermo Fisher Scientific LTQ FT Ultra (ESI-MS) and Waters Micromass GCT Premier (EI-MS).

### Example 1: (E)-2-methyl-N-(4-methylbenzyl)undecan-1-imine oxide

a) 4-Methylbenzaldehyde oxime (215 mmol, 29.00 g, 1 equiv) was dissolved in 70 mL MeOH. NaBH₃CN (257 mmol, 16.18 g, 1.2 equiv) was added in portions at 0° C. Conc. HCl (279 mmol, 27.5 g, 1.3 equiv) in 50 mL MeOH was added dropwise at 0° C. After the addition the pH was <4. The reaction was stirred at r.t. overnight, and the pH was still <4. GC and TLC monitoring was used to ensure that no oxime is left. The solvent was removed and 10 mL MTBE and 100 mL water was added to solve the residue. The water layer was saved and then washed by 10 mL MTBE. The water phase was saved, and solvent was removed under vacuum to obtain the residue (purity about 71.2%) as a white solid which was used for next step without further purification. The purity of the product was calculated as it's a mixture of product and NaCl (m/m=1:1.2).

b) 10% aq. sodium hydroxide (45.60 g, 114 mmol, 1.1 equiv) in 112 mL water and N-(4-methylbenzyl)hydroxylamine hydrochloride (25.27 g, 102 mmol, 1 equiv) was stirred at r.t. under Argon atmosphere, pH>7 and stirred for another 5 minutes. Dichloromethane (100 ml) was added to extracted the free hydroxylamine, then the organic layer was combined and added dropwise to a mixture of 2-methylundecanal (21.01 g, 114 mmol, 1.1 equiv) in Dichloromethane (100 ml). The mixture was stirred at r.t. for 2 hours. The reaction progress was monitored by TLC and GC-MS. After no further conversion could be observed, the mixture was dried and solvent removed followed by silica gel chromatography (hep-tane:MTBE=20:1-0:1) to obtain (E)-2-methyl-N-(4-methylbenzyl)undecan-1-imine oxide (25.37 g, 82 mmol, 79% yield) as a white solid, which was recrystallized from hexane and DCM (10:1) to give 21.53 g pure product as a white solid.

¹H NMR (400 MHz, CDCl₃) δ 7.27 (d, J=7.7 Hz, 2H), 7.19 (d, J=7.7 Hz, 2H), 6.42 (d, J=7.5 Hz, 1H), 4.82 (s, 2H), 3.19-2.95 (m, 1H), 2.36 (s, 3H), 1.44-1.19 (m, 16H), 1.04 (d, J=6.9 Hz, 3H), 0.88 (t, J=6.7 Hz, 3H). ¹³C NMR (101 MHz, CDCl₃) δ 144.03 (t), 138.80 (q), 130.24 (q), 129.68 (t), 129.27 (t), 69.23 (d), 34.12, 31.99, 31.06 (t), 29.69 (d), 29.63 (d), 29.41 (d), 27.26 (d), 22.78 (d), 21.31 (s), 16.58 (s), 14.21 (s). GC/MS (EI): m/z (%): 303 (2) [M⁺], 286 (2), 260 (2), 207 (5), 177 (7), 161 (18), 105 (100), 77 (13), 55 (12).

### Example 2: (1Z,2E)-N-benzylnon-2-en-1-imine oxide

The compound was obtained from N-benzylhydroxylamine hydrochloride (4.7 mmol, 0.75 g, 1 equiv) and (E)-non-2-enal (5.2 mmol, 0.73 g, 1.1 equiv) according to the process of example 1 as white solid (2.2 mmol, 0.57 g, 47% yield).

¹H NMR (400 MHz, CDCl3) δ 7.49-7.28 (m, 5H), 6.98 (d, J=9.4 Hz, 1H), 6.75 (dd, J=15.9, 9.4 Hz, 1H), 6.30-6.08 (m, 1H), 4.88 (s, 2H), 2.32-2.08 (m, 2H), 1.48-1.35 (m, 2H), 1.32-1.21 (m, 6H), 0.86 (t, J=6.4 Hz, 3H). ¹³C NMR (101 MHz, CDCl3) δ 143.91 (t), 136.51 (t), 133.17 (q), 129.40 (t), 129.06 (t), 129.00 (t), 121.10 (t), 69.05 (d), 33.47 (d), 31.73 (d), 28.97 (d), 28.68 (d), 22.67 (d), 14.18 (s). GC/MS (EI): m/z (%): 245 (1) [M+], 229 (5), 186 (6), 172 (15), 144 (22), 104 (17), 92 (16), 91 (100), 65 (12).

### Example 3: (E)-N-cyclohexyl-1-(4-(4-hydroxy-4-methylpentyl)cyclohex-3-en-1-yl)methanimine oxide

The compound was obtained from 4-(4-hydroxy-4-methylpentyl)cyclohex-3-ene-1-carbaldehyde (24.8 mmol, 5.22 g, 1.1 equiv) and N-cyclohexylhydroxylamine (22.5 mmol, 2.55 g, 1 equiv) according to the process of example 1 as white solid (6.7 mmol, 2.10 g, 30% yield).

¹H NMR (400 MHz, CDCl3) δ 6.59 (d, J=6.1 Hz, 1H), 5.61-5.21 (m, 1H), 3.79-3.45 (m, 1H), 3.32-2.99 (m, 1H), 2.40-2.15 (m, 1H), 2.08-1.79 (m, 12H), 1.70-1.38 (m, 7H), 1.34-1.26 (m, 2H), 1.23-1.16 (m, 7H). ¹³C NMR (101 MHz, CDCl3) δ 140.22 (t), 137.74 (q), 119.07 (t), 73.96 (t), 70.91 (q), 43.45 (d), 38.04 (d), 31.23 (d), 31.13 (d), 30.83 (s), 29.35 (d), 29.23 (d), 28.01 (d), 26.91 (d), 25.04 (d), 24.86 (d), 22.27 (d). GC/MS (EI): m/z (%): 307 (1) [M+], 287 (29), 258 (25), 190 (30), 186 (62), 130 (68), 117 (100), 91 (81), 59 (80).

### Example 4: (E)-N-benzyl-2-methylundecan-1-imine oxide

The compound was obtained from 2-methylundecanal (14.1 mmol, 2.59 g, 1.1 equiv and N-benzylhydroxylamine hydrochloride (12.8 mmol, 2.10 g, 1 equiv) according to the process of example 1 as white solid (11.2 mmol, 3.48 g, 88% yield).

¹H NMR (400 MHz, CDCl3) δ 7.49-7.29 (m, 5H), 6.48 (d, J=7.5 Hz, 1H), 4.86 (s, 2H), 3.18-2.95 (m, 1H), 1.58-1.11 (m, 16H), 1.05 (d, J=6.9 Hz, 3H), 0.88 (t, J=6.7 Hz, 3H). ¹³C NMR (101 MHz, CDCl3) δ 144.20 (t), 133.25 (q), 129.06 (t), 128.88 (t), 128.78 (t), 69.41 (d), 34.02 (d), 31.89 (d), 31.00 (t), 29.59 (d), 29.53 (d), 29.31 (d), 27.18 (d), 22.68 (d), 16.49 (s), 14.12 (s). GC/MS (EI): m/z (%): 289 (3) [M+], 272 (5), 247 (5), 176 (5), 163 (25), 147 (7), 91 (100), 65 (8), 55 (5).

### Example 5: (E)-N-benzyl-2-methylundecan-1-imine oxide

Alternatively, the N-benzylhydroxylamine hydrochloride for the preparation of the title compound was prepared as follows:

a) (E)-N-benzyl-1-phenylmethanimine oxide

Dibenzylamine (3.51 g, 17.6 mmol) was placed in a 100 mL three-necked round-bottomed flask, and Na₂WO₄·2H₂O (0.063 g, 0.176 mmol) and methanol (50 ml) were added. The solution was cooled, and 30% aq. hydrogen peroxide (3.99 g, 35.2 mmol) was added dropwise under stirring over a period of 3 mins. After stirring at room temperature overnight, the total conversion of dibenzylamine was confirmed by TLC, GC-MS and NMR. The solvent was removed, and the residue was purified by column chromatography (Heptane:MTBE=4:1-1:1) to obtain (E)-N-benzyl-1-phenylmethanimine oxide (3.38 g, 15.5 mmol, 88% yield) as a white solid.

¹H NMR (400 MHz, CDCl₃) δ 8.25-8.16 (m, 2H), 7.50-7.42 (m, 2H), 7.42-7.30 (m, 7H), 5.01 (s, 2H). ¹³C NMR (101 MHz, CDCl₃) δ 134.32 (t), 133.35 (q), 130.49 (t), 129.23 (t), 128.98 (t), 128.65 (t), 128.49 (t), 71.25 (d). GC/MS (EI): m/z (%): 211 (28) [M⁺], 181 (25), 92 (35), 91 (100), 89 (28), 77 (12), 65 (72), 63 (18).

b) N-benzylhydroxylamine hydrochloride

(E)-N-benzyl-1-phenylmethanimine oxide (2.012 g, 9.52 mmol) in DCM (20 ml) was added into the flask, and then 37% aq. hydrogen chloride (1.877 g, 19.05 mmol) was added dropwise at room temperature. Then the reaction mixture was stirred at reflux for 2 hours. The phases have been separated, the water layer was collected, and the organic phase was washed with water twice. The water layers were combined, and water was removed to obtain N-benzylhydroxylamine hydrochloride (1.25 g, 7.7 mmol, 81% yield) as white solid, which was used for next step without further purification.

¹H NMR (400 MHz, DMSO) δ 11.87 (s, 2H), 10.99 (s, 1H), 7.60-7.48 (m, 2H), 7.46-7.30 (m, 3H), 4.30 (s, 2H). ¹³C NMR (101 MHz, DMSO) δ 131.29 (t), 130.41 (q), 129.47 (t), 128.88 (t), 54.23 (d).

c) (E)-N-benzyl-2-methylundecan-1-imine oxide

The last step was carried out as described in Example 4.

### Example 6: (E)-N-cyclohexyl-2-methylundecan-1-imine oxide

The compound was obtained from 2-methylundecanal (164.0 mmol, 30.20 g, 1.1 equiv) and N-cyclohexylhydroxylamine (149.0 mmol, 16.84 g, 1 equiv) according to the process of example 1 as white solid (87.0 mmol, 25.00 g, 59% yield).

¹H NMR (400 MHz, CDCl3) δ 6.51 (d, J=7.5 Hz, 1H), 3.73-3.48 (m, 1H), 3.22-2.95 (m, 1H), 2.09-1.94 (m, 2H), 1.92-1.79 (m, 4H), 1.67 (d, J=12.3 Hz, 1H), 1.47-1.17 (m, 19H), 1.06 (d, J=6.8 Hz, 3H), 0.88 (t, J=6.6 Hz, 3H). ¹³C NMR (101 MHz, CDCl3) δ 141.60 (t), 73.97 (t), 34.25 (d), 31.91 (d), 31.26 (d), 31.11 (d), 30.58 (t), 29.68 (d), 29.58 (d), 29.32 (d), 27.21 (d), 25.09 (d), 25.06 (d), 25.04 (d), 22.69 (d), 16.70 (s), 14.13 (s). GC/MS (EI): m/z (%): 281 (8) [M+], 264 (10), 239 (12), 168 (16), 155 (76), 139 (32), 126 (19), 83 (46), 55 (100).

### Example 7: (Z)-2-methyl-N-phenylundecan-1-imine oxide

The compound was obtained from 2-methylundecanal (20.2 mmol, 3.72 g, 1.1 equiv) and N-phenylhydroxylamine (18.3 mmol, 4.00 g, 1 equiv) according to the process of example 1 as white solid (12.1 mmol, 3.50 g, 66% yield).

¹H NMR (400 MHz, CDCl3) δ 7.77-7.57 (m, 2H), 7.53-7.32 (m, 3H), 7.05 (d, J=7.6 Hz, 1H), 3.44-3.19 (m, 1H), 1.68-1.45 (m, 2H), 1.42-1.16 (m, 17H), 0.88 (t, J=6.5 Hz, 3H). ¹³C NMR (101 MHz, CDCl3) δ 147.93 (q), 144.62 (t), 129.78 (t), 129.01 (t), 121.71 (t), 34.18 (d), 31.89 (d), 31.70 (t), 29.69 (d), 29.57 (d), 29.31 (d), 27.34 (d), 22.68 (d), 16.55 (s), 14.12 (s). GC/MS (EI): m/z (%): 275 (2) [M+], 259 (2), 232 (98), 172 (16), 146 (86), 133 (100), 120 (90), 106 (75), 77 (71), 55 (23).

### Example 8: (E)-N-cyclopentyl-2-methylundecan-1-imine oxide

The compound was obtained from 2-methylundecanal (23.9 mmol, 4.40 g, 1.1 equiv) and N-cyclopentylhydroxylamine (21.7 mmol, 2.15 g, 1 equiv) according to the process of example 1 as white solid (16.7 mmol, 4.69 g, 77% yield).

¹H NMR (400 MHz, CDCl3) δ 6.58 (d, J=7.5 Hz, 1H), 4.49-4.02 (m, 1H), 3.34-2.89 (m, 1H), 2.26-2.07 (m, 2H), 2.01-1.77 (m, 4H), 1.69-1.51 (m, 2H), 1.47-1.19 (m, 16H), 1.06 (d, J=6.9 Hz, 3H), 0.88 (t, J=6.7 Hz, 3H). ¹³C NMR (101 MHz, CDCl3) δ 142.42 (t), 74.71 (t), 34.16 (d), 31.90 (d), 31.26 (d), 31.13 (d), 30.81 (t), 29.66 (d), 29.57 (d), 29.56 (d), 29.31 (d), 27.22 (d), 25.52 (d), 22.68 (d), 16.62 (s), 14.12 (s). GC/MS (EI): m/z (%): 267 (10) [M+], 250 (12), 225 (15), 200 (7), 154 (32), 141 (100), 86 (26), 69 (51), 55 (28).

### Example 9: (E)-N-isopropyl-2-methylundecan-1-imine oxide

A mixture of N-isopropylhydroxylamine (30.6 g, 61 mmol, 1 equiv) (15% w/w aqueous) and methylundecanal (11.26 g, 61 mmol, 1 equiv) was stirred vigorously at r.t. for 2 hours.

The conversion was monitored by TLC. After conversion of >95%, the solvent was removed, followed by silica gel chromatography (heptane:MTBE=20:1-0:1) to give a crude product as a white solid, which was recrystallized with Hexane and DCM(10:1) to obtain (E)-N-isopropyl-2-methylundecan-1-imine oxide (14.254 g, 56.1 mmol, 92% yield) as a white solid.

¹H NMR (400 MHz, CDCl3) δ 6.52 (d, J=7.4 Hz, 1H), 4.07-3.79 (m, 1H), 3.28-2.88 (m, 1H), 1.49-1.19 (m, 22H), 1.07 (d, J=6.5 Hz, 3H), 0.87 (d, J=5.7 Hz, 3H). ¹³C NMR (101 MHz, CDCl3) δ 141.22 (t), 66.15 (t), 34.16 (d), 31.88 (d), 30.58 (t), 29.64 (d), 29.55 (d), 29.29 (d), 27.19 (d), 22.66 (d), 20.89 (s), 20.75 (s), 16.60 (s), 14.09 (s). GC/MS (EI): m/z (%): 241 (7) [M+], 226 (7), 199 (11), 184 (11), 128 (36), 115 (100), 100 (25), 86(18), 73 (16), 55 (22).

### Example 10: (2E,3E)-N-isopropyl-4-(2,6,6-trimethylcyclohex-1-en-1-yl)but-3-en-2-imine oxide

The compound was obtained from β-Ionone (57.2 mmol, 6.38 g, 1.1 equiv) and N-isopropylhydroxylamine hydrochloride (52.0 mmol, 10.0 g, 1 equiv) according to the process of example 9 as yellow oil (13.8 mmol, 3.50 g, 26% yield).

¹H NMR (400 MHz, CDCl3) δ 6.60-6.26 (m, 2H), 4.79-4.54 (m, 1H), 2.26 (s, 3H), 2.05 (t, J=6.2 Hz, 2H), 1.74 (s, 3H), 1.68-1.57 (m, 2H), 1.53-1.46 (m, 2H), 1.39 (d, J=6.5 Hz, 6H), 1.04 (s, 6H). ¹³C NMR (101 MHz, CDCl3) δ 143.59 (q), 137.54 (q), 132.69 (t), 131.44 (q), 123.82 (t), 57.12 (t), 39.38 (d), 34.20 (q), 33.11 (d), 28.91 (s), 21.76 (s), 20.26 (s), 19.04 (d), 13.92 (s). GC/MS (EI): m/z (%): 249 (1) [M+], 234 (3), 216 (1), 207 (2), 192 (100), 160 (30), 146 (4), 134 (6), 105 (9).

### Example 11: (E)-2-methyl-N-(3-methylbut-2-en-1-yl)undecan-1-imine oxide

The compound was obtained from 2-methylundecanal (12.7 mmol, 2.35 g, 1.1 equiv) and N-(3-methylbut-2-en-1-yl)hydroxylamine (11.6 mmol, 1.15 g, 1 equiv) according to the process of example 1 as colorless oil (3.0 mmol, 0.85 g, 26% yield).

¹H NMR (400 MHz, CDCl₃) δ 6.49 (d, J=7.5 Hz, 1H), 5.49-5.31 (m, 1H), 4.34 (d, J=7.3 Hz, 2H), 3.21-2.95 (m, 1H), 1.82 (s, 3H), 1.72 (s, 3H), 1.34-1.20 (m, 15H), 1.07 (d, J=6.9 Hz, 3H), 0.98-0.83 (m, 4H). ¹³C NMR (101 MHz, CDCl₃) δ 143.16 (t), 141.04 (q), 116.44 (t), 62.97 (d), 34.20 (d), 32.00 (d), 31.00 (t), 29.75 (d), 29.66 (d), 29.42 (d), 27.29 (d), 25.92 (s), 22.78 (d), 18.21 (s), 16.66 (s), 14.21 (s). GC/MS (EI): m/z (%): 267 (1) [M⁺], 252 (1), 196 (1), 180 (1), 156 (4), 141 (2), 114 (11), 69 (100), 55 (15).

### Example 12: (Z)—N-(furan-2-ylmethyl)-2-methylundecan-1-imine oxide

The compound was obtained from 2-methylundecanal (13.7 mmol, 2.53 g, 1.1 equiv) and N-(furan-2-ylmethyl)hydroxylamine (12.5 mmol, 1.87 g, 1 equiv) according to the process of example 1 as white solid (8.3 mmol, 2.39 g, 67% yield).

¹H NMR (400 MHz, CDCl₃) δ 7.49-7.40 (m, 1H), 6.51 (d, J=3.2 Hz, 1H), 6.43-6.39 (m, 1H), 6.37 (d, J=7.6 Hz, 1H), 4.88 (s, 2H), 3.23-2.95 (m, 1H), 1.34-1.16 (m, 16H), 1.06 (d, J=6.9 Hz, 3H), 0.88 (t, J=6.8 Hz, 3H). ¹³C NMR (101 MHz, CDCl₃) δ 146.31 (q), 143.97 (t), 143.68 (t), 112.06 (t), 110.98 (t), 61.48 (d), 34.00 (d), 31.89 (d), 31.03 (t), 29.58 (d), 29.52 (d), 29.30 (d), 27.09 (d), 22.67 (d), 16.43 (s), 14.11 (s). GC/MS (EI): m/z (%): 279 (1) [M+], 261 (1), 236 (1), 204 (1), 180 (6), 162 (4), 137 (5), 81 (100), 69 (5).

### Example 13: (E)-N,2-dimethylundecan-1-imine oxide

The compound was obtained from 2-methylundecanal (39.7 mmol, 7.32 g, 1.1 equiv) and N-Methylhydroxylamine hydrochloride (36.1 mmol, 3.01 g, 1 equiv) according to the process of example 1 as white solid (39.7 mmol, 7.50 g, 94% yield).

¹H NMR (400 MHz, CDCl₃) δ 6.51 (dd, J=7.6, 0.6 Hz, 1H), 3.82-3.45 (m, 3H), 3.22-2.84 (m, 1H), 1.49-1.18 (m, 16H), 1.08 (d, J=6.9 Hz, 3H), 0.88 (t, J=6.9 Hz, 3H). ¹³C NMR (101 MHz, CDCl₃) δ 145.07 (t), 52.61 (t), 33.95 (d), 31.83 (d), 31.10 (t), 29.59 (d), 29.51 (d), 29.49 (d), 29.25 (d), 27.12 (d), 22.62 (d), 16.35 (s), 14.05 (s). GC/MS (EI): m/z (%): 213 (4) [M⁺], 171 (5), 142 (2), 114 (4), 100 (46), 87 (100), 70 (45), 60 (36), 55 (70).

### Example 14: (E)-N-ethyl-2-methylundecan-1-imine oxide

The compound was obtained from 2-methylundecanal (10.9 mmol, 1.36 g, 1.1 equiv) and N-ethylhydroxylamine (9.9 mmol, 0.58 g, 1 equiv) according to the process of example 1 as white solid (6.9 mmol, 1.60 g, 70% yield).

¹H NMR (400 MHz, CDCl₃) δ 6.52 (d, J=7.5 Hz, 1H), 3.79 (q, J=7.2 Hz, 2H), 3.19-2.90 (m, 1H), 1.52-1.42 (m, 4H), 1.35-1.18 (m, 15H), 1.08 (d, J=6.8 Hz, 3H), 0.88 (t, J=6.4 Hz, 3H). ¹³C NMR (101 MHz, CDCl₃) δ 143.27 (t), 60.28 (d), 34.10 (d), 31.90 (d), 30.88 (t), 29.67 (d), 29.57 (d), 29.33 (d), 27.19 (d), 22.69 (d), 16.54 (s), 14.14 (s), 13.53 (s). GC/MS (EI): m/z (%): 227 (1) [M⁺], 210 (2), 185 (4), 170 (3), 128 (3), 114 (40), 101 (100), 84 (27), 55 (35).

### Example 15: (E)-2-methyl-N-(2-methylpentyl)undecan-1-imine oxide (mixture of diastereoisomers)

The compound was obtained from 2-methylundecanal (50.4 mmol, 9.30 g, 1.1 equiv) and N-(2-methylpentyl)hydroxylamine (45.8 mmol, 5.27 g, 1 equiv) according to the process of example 1 as colorless oil (24.7 mmol, 7.36 g, 54% yield).

¹H NMR (400 MHz, CDCl₃) δ 6.47 (d, J=7.5 Hz, 1H), 3.75-3.57 (m, 1H), 3.51-3.34 (m, 1H), 3.20-2.95 (m, 1H), 2.47-2.21 (m, 1H), 1.54-1.12 (m, 20H), 1.09 (d, J=6.9 Hz, 3H), 0.95-0.82 (m, 9H). ¹³C NMR (101 MHz, CDCl₃) δ 144.45 (t), 71.68 (d), 71.65 (d), 36.10 (d), 34.07 (d), 33.99 (d), 31.80 (d), 30.83 (t), 30.40 (t), 30.39 (t), 29.57 (d), 29.54 (d), 29.47 (d), 29.22 (d), 27.22 (d), 22.58 (d), 19.69 (d), 19.67 (d), 16.84 (s), 16.75 (s), 16.52 (s), 14.08 (s), 14.07 (s), 14.00 (s). GC/MS (EI): m/z (%): 283 (2) [M⁺], 266 (5), 241 (5), 212 (10), 170 (20), 157 (20), 128 (100), 112 (11), 86 (21).

### Example 16: (E)-N-(benzo[d][1,3]dioxol-5-ylmethyl)-2-methylundecan-1-imine oxide

The compound was obtained from 2-methylundecanal (16.9 mmol, 3.11 g, 1.1 equiv) and N-(benzo[d][1,3]dioxol-5-ylmethyl)-hydroxylamine (15.3 mmol, 2.53 g, 1 equiv) according to the process of example 1 as white solid (12.6 mmol, 4.20 g, 82% yield).

¹H NMR (400 MHz, CDCl₃) δ 6.89 (s, 1H), 6.83 (dd, J=16.9, 7.8 Hz, 2H), 6.45 (d, J=7.4 Hz, 1H), 5.98 (s, 2H), 4.75 (s, 2H), 3.17-2.91 (m, 1H), 1.45-1.18 (m, 16H), 1.05 (d, J=6.4 Hz, 3H), 0.87 (d, J=6.7 Hz, 3H). ¹³C NMR (101 MHz, CDCl₃) δ 148.16 (q), 148.06 (q), 143.93 (t), 126.77 (q), 123.22 (t), 109.58 (t), 108.50 (t), 101.33 (d), 69.15 (d), 34.02 (d), 31.89 (d), 30.99 (t), 29.61 (d), 29.54 (d), 29.32 (d), 27.19 (d), 22.69 (d), 16.49 (s), 14.12 (s). GC/MS (EI): m/z (%): 333 (1) [M⁺], 315 (13), 272 (4), 258 (11), 216 (28), 202 (100), 172 (14), 148 (86), 135 (60).

### Example 17: 2-methyl-N-(naphthalen-2-ylmethyl)undecan-1-imine oxide

The compound was obtained from 2-methylundecanal (18.98 mmol, 3.50 g, 1.1 equiv) and N-(naphthalen-2-ylmethyl)hydroxylamine (17.25 mmol, 2.95 g, 1 equiv) according to the process of example 1 as white solid (15.0 mmol, 5.20 g, 87% yield).

¹H NMR (400 MHz, CDCl₃) δ 7.86 (s, 4H), 7.56-7.37 (m, 3H), 6.50 (d, J=7.3 Hz, 1H), 5.03 (s, 2H), 3.24-2.95 (m, 1H), 1.51-1.14 (m, 16H), 1.06 (d, J=6.4 Hz, 3H), 0.87 (d, J=6.8 Hz, 3H). ¹³C NMR (101 MHz, CDCl₃) δ 144.37 (t), 133.33 (q), 133.28 (q), 130.60 (q), 128.82 (t), 128.78 (t), 128.09 (t), 127.75 (t), 126.63 (t), 126.51 (t), 126.21 (t), 69.61 (d), 34.05 (d), 31.89 (d), 31.07 (t), 29.61 (d), 29.54 (d), 29.32 (d), 27.22 (d), 22.70 (d), 16.54 (s), 14.13 (s). GC/MS (EI): m/z (%): 339 (1) [M⁺], 321 (18), 264 (19), 222 (30), 208 (94), 154 (81), 141 (100), 127 (24), 81 (18).

### Example 18: (Z)-2-methyl-N-(2-methylundecyl)undecan-1-imine oxide (mixture of diastereoisomers)

The compound was obtained from 2-methylundecanal (16.3 mmol, 3.00 g, 1.1 equiv) and N-(2-methylundecyl)hydroxylamine (14.8 mmol, 2.95 g, 1 equiv) according to the process of example 1 as white solid (10.9 mmol, 4.01 g, 74% yield).

¹H NMR (400 MHz, CDCl₃) δ 6.45 (d, J=7.5 Hz, 1H), 3.80-3.55 (m, 1H), 3.49-3.36 (m, 1H), 3.23-2.97 (m, 1H), 2.42-2.19 (m, 1H), 1.37-1.19 (m, 32H), 1.08 (d, J=6.8 Hz, 3H), 0.96-0.75 (m, 10H). ¹³C NMR (101 MHz, CDCl₃) δ 144.83 (t), 71.80 (d), 71.78 (d), 34.17 (d), 34.09 (d), 33.97 (d), 31.90 (d), 30.95 (t), 30.71 (t), 29.82 (d), 29.77 (d), 29.69 (d), 29.64 (d), 29.59 (d), 29.33 (d), 27.32 (d), 27.29 (d), 26.61 (d), 22.68 (d), 17.00 (s), 16.91 (s), 16.62 (s), 14.11 (s). GC/MS (EI): m/z (%): 367 (1) [M⁺], 334 (1), 294 (1), 264 (3), 250 (6), 225 (6), 196 (100), 180 (18), 154 (8).

### Example 19: (E)-N-(4-methoxybenzyl)-2-methylundecan-1-imine oxide

The compound was obtained from 2-methylundecanal (12.7 mmol, 2.34 g, 1.1 equiv) and N-(4-methoxybenzyl)hydroxylamine (11.5 mmol, 1.74 g, 1 equiv) according to the process of example 1 as white solid (8.2 mmol, 2.62 g, 71% yield).

¹H NMR (400 MHz, CDCl₃) δ 7.31 (d, J=8.5 Hz, 2H), 6.91 (d, J=8.5 Hz, 2H), 6.41 (d, J=7.5 Hz, 1H), 4.79 (s, 2H), 3.82 (s, 3H), 3.15-2.95 (m, 1H), 1.42-1.18 (m, 16H), 1.04 (d, J=6.9 Hz, 3H), 0.88 (t, J=6.8 Hz, 3H). ¹³C NMR (101 MHz, CDCl₃) δ 160.03 (q), 143.86 (t), 130.77 (t), 125.17 (q), 114.31 (t), 68.80 (d), 55.32 (s), 34.03 (d), 31.90 (d), 30.96 (t), 29.60 (d), 29.54 (d), 29.32 (d), 27.17 (d), 22.69 (d), 16.50 (s), 14.12 (s). GC/MS (EI): m/z (%): 319 (1) [M+], 274 (1), 255 (3), 176 (2), 146 (5), 121 (100), 108 (4), 91 (14), 77 (35).

### Example 20: (E)-3-(4-isobutyl-2-methylphenyl)-N-methylpropan-1-imine oxide

The compound was obtained from 3-(4-isobutyl-2-methylphenyl)propanal (16.0 mmol, 3.27 g, 1.1 equiv) and methylhydroxylamine hydrochloride (14.6 mmol, 1.22 g, 1 equiv) according to the process of example 1 as light yellow oil (13.2 mmol, 3.27 g, 91% yield).

¹H NMR (400 MHz, CDCl₃) δ 7.05 (d, J=5.9 Hz, 1H), 6.95-6.89 (m, 2H), 6.69 (t, J=5.2 Hz, 1H), 3.66 (s, 3H), 2.85-2.64 (m, 4H), 2.40 (d, J=7.1 Hz, 2H), 2.30 (s, 4H), 0.89 (d, J=6.7 Hz, 6H). ¹³C NMR (101 MHz, CDCl₃) δ 139.84 (q), 139.56 (t), 135.67 (q), 135.66 (q), 131.23 (t), 128.91 (t), 128.14 (t), 126.84 (t), 52.41 (s), 44.98 (d), 30.19 (t), 28.15 (d), 26.98 (d), 22.44 (s), 19.30 (s). GC/MS (EI): m/z (%): 233 (6) [M⁺], 216 (29), 187 (40), 161 (100), 143 (27), 128 (31), 119 (71), 105 (30), 91 (38).

### Example 21: (E)-N-benzyl-3-(4-isobutyl-2-methylphenyl)propan-1-imine oxide

The compound was obtained from 3-(4-isobutyl-2-methylphenyl)propanal (14.2 mmol, 2.90 g, 1.1 equiv) and N-benzylhydroxylamine hydrochloride (12.9 mmol, 2.06 g, 1 equiv) according to the process of example 1 as white solid (10.6 mmol, 3.49 g, 82% yield).

¹H NMR (400 MHz, CDCl₃) δ 7.43-7.28 (m, 5H), 6.96-6.85 (m, 3H), 6.64 (t, 1H), 4.85 (s, 2H), 2.82-2.71 (m, 4H), 2.40 (d, J=7.1 Hz, 2H), 2.25 (s, 3H), 1.90-1.77 (m, 1H), 0.89 (d, J=6.6 Hz, 6H). ¹³C NMR (101 MHz, CDCl₃) δ 139.79 (q), 138.59 (t), 135.71 (q), 135.66 (q), 132.82 (q), 131.19 (t), 129.43 (t), 128.94 (t), 128.30 (t), 126.80 (t), 69.27 (d), 45.00 (d), 30.23 (t), 28.19 (d), 26.92 (d), 22.47 (s), 19.31 (s). GC/MS (EI): m/z (%): 309 (3) [M⁺], 292 (8), 250 (1), 187 (14), 161 (42), 143 (6), 131 (10), 119 (19), 91 (100).

### Example 22: (E)-N-ethyl-3-(4-isobutyl-2-methylphenyl)propan-1-imine oxide

The compound was obtained from 3-(4-isobutyl-2-methylphenyl)propanal (23.3 mmol, 4.76 g, 1.1 equiv) and ethylhydroxylamine (21.2 mmol, 1.25 g, 1 equiv) according to the process of example 1 as light yellow oil (12.3 mmol, 3.26 g, 58% yield).

¹H NMR (400 MHz, CDCl₃) δ 7.03 (d, J=7.6 Hz, 1H), 6.96-6.90 (m, 2H), 6.73 (t, J=5.3 Hz, 1H), 3.79 (q, J=7.3 Hz, 2H), 2.87-2.71 (m, 5H), 2.50-2.35 (m, 3H), 2.33-2.24 (m, 4H), 1.87-1.79 (m, 1H), 1.45 (t, J=7.3 Hz, 3H), 0.89 (d, J=6.6 Hz, 6H). ¹³C NMR (101 MHz, CDCl₃) δ 139.80 (q), 137.98 (t), 135.70 (q), 135.66 (q), 131.20 (t), 128.19 (t), 126.80 (t), 60.04 (d), 44.97 (d), 30.18 (t), 28.22 (d), 26.78 (d), 22.43 (s), 19.31 (s), 13.32 (s). GC/MS (EI): m/z (%): 247 (7) [M⁺], 230 (15), 187 (34), 161 (100), 143 (10), 131 (17), 119 (50), 105 (18), 91 (18).

### Example 23: (E)-3-(4-isobutyl-2-methylphenyl)-N-(4-methoxybenzyl)propan-1-imine oxide

The compound was obtained from 3-(4-isobutyl-2-methylphenyl)propanal (33.9 mmol, 7.28 g, 1.1 equiv) and N-(4-methoxybenzyl)hydroxylamine (30.8 mmol, 4.65 g, 1 equiv) according to the process of example 1 as white solid (21.65 mmol, 7.5 g, 70% yield).

¹H NMR (400 MHz, CDCl₃) δ 7.37-7.18 (m, 2H), 6.98-6.79 (m, 5H), 6.59 (t, J=5.3 Hz, 1H), 4.80 (s, 2H), 3.82 (s, 3H), 2.85-2.63 (m, 4H), 2.40 (d, J=7.1 Hz, 2H), 2.25 (s, 3H), 1.84-1.79 (m, 1H), 0.89 (d, J=6.6 Hz, 6H). ¹³C NMR (101 MHz, CDCl₃) δ 160.11 (q), 139.77 (q), 138.16 (t), 135.75 (q), 135.65 (q), 131.17 (t), 131.06 (t), 128.30 (t), 126.76 (t), 124.77 (q), 114.31 (t), 68.64 (d), 55.35 (s), 44.98 (d), 30.23 (t), 28.21 (d), 26.89 (d), 22.45 (s), 19.31 (s). GC/MS (EI): m/z (%): 339 (1) [M⁺], 323 (2), 280 (1), 202 (9), 185 (3), 162 (6), 121 (100), 91 (8), 78 (7).

### Example 24: (E)-3-(4-isobutyl-2-methylphenyl)-N-isopropylpropan-1-imine oxide

The compound was obtained from 3-(4-isobutyl-2-methylphenyl)propanal (18.7 mmol, 4.03 g, 1 equiv) and N-isopropylhydroxylamine (15% aq. solution, 18.7 mmol, 9.37 g, 1 equiv) according to the process of example 9 as white solid (9.6 mmol, 2.66 g, 51% yield).

¹H NMR (300 MHz, CDCl₃) δ 7.10-6.99 (m, 1H), 6.97-6.84 (m, 2H), 6.73 (t, J=5.3 Hz, 1H), 4.12-3.92 (m, 1H), 2.93-2.64 (m, 4H), 2.40 (d, J=7.1 Hz, 2H), 2.31 (s, 3H), 1.90-1.74 (m, 1H), 1.40 (d, J=6.5 Hz, 6H), 0.89 (d, J=6.6 Hz, 6H). ¹³C NMR (75 MHz, CDCl₃) δ 139.86 (q), 135.96 (q), 135.81 (q), 135.70 (t), 131.27 (t), 128.34 (t), 126.86 (t), 66.20 (t), 45.08 (d), 30.29 (t), 28.44 (d), 26.65 (d), 22.54 (s), 20.87 (s), 19.45 (s). GC/MS (EI): m/z (%): 261 (10) [M⁺], 244 (10), 230 (5), 202 (4), 187 (36), 161 (100), 131 (17), 119 (48), 105 (19).

### Example 25: (E)-N-cyclohexyl-3-(4-isobutyl-2-methylphenyl)propan-1-imine oxide

The compound was obtained from 3-(4-isobutyl-2-methylphenyl)propanal (16.2 mmol, 3.31 g, 1.1 equiv) and N-cyclohexylhydroxylamine (14.7 mmol, 1.66 g, 1 equiv) according to the process of example 1 as white solid (11.3 mmol, 3.59 g, 77% yield).

¹H NMR (400 MHz, CDCl₃) δ 7.07-6.99 (m, 1H), 6.98-6.83 (m, 2H), 6.72 (t, J=5.4 Hz, 1H), 3.75-3.50 (m, 1H), 2.86-2.69 (m, 4H), 2.40 (d, J=7.2 Hz, 2H), 2.30 (s, 3H), 2.01-1.92 (m, 2H), 1.90-1.74 (m, 5H), 1.66 (d, J=12.2 Hz, 1H), 1.36-1.07 (m, 3H), 0.89 (d, J=6.6 Hz, 6H). ¹³C NMR (101 MHz, CDCl₃) δ 139.72 (q), 136.13 (t), 135.86 (q), 135.69 (q), 131.16 (t), 128.23 (t), 126.76 (t), 73.81 (t), 44.98 (d), 31.04 (d), 30.18 (t), 28.35 (d), 26.57 (d), 25.06 (d), 25.00 (d), 22.43 (s), 19.35 (s). GC/MS (EI): m/z (%): 301 (6) [M⁺], 284 (13), 242 (6), 207 (14), 187 (27), 161 (100), 131 (21), 119 (40), 83 (20).

### Example 26: (E)-N-(benzo[d][1,3]dioxol-5-ylmethyl)-3-(4-isobutyl-2-methylphenyl)propan-1-imine oxide

The compound was obtained from 3-(4-isobutyl-2-methylphenyl)propanal (32.1 mmol, 6.91 g, 1.1 equiv) and N-(benzo[d][1,3]dioxol-5-ylmethyl)hydroxylamine (29.2 mmol, 4.82 g, 1 equiv) according to the process of example 1 as white solid (19.8 mmol, 7.01 g, 68% yield).

¹H NMR (400 MHz, CDCl₃) δ 6.99-6.93 (m, 1H), 6.93-6.77 (m, 5H), 6.63 (t, J=5.1 Hz, 1H), 5.98 (s, 2H), 4.75 (s, 2H), 2.86-2.69 (m, 4H), 2.40 (d, J=7.2 Hz, 2H), 2.31-2.23 (m, 3H), 1.85-1.79 (m, 1H), 0.89 (d, J=6.5 Hz, 6H). ¹³C NMR (101 MHz, CDCl₃) b 148.25 (q), 148.07 (q), 139.83 (q), 138.28 (t), 135.71 (q), 135.66 (q), 131.19 (t), 128.29 (t), 126.78 (t), 126.33 (q), 123.55 (t), 109.85 (t), 108.52 (t), 101.38 (d), 68.99 (d), 44.98 (d), 30.22 (t), 28.21 (d), 26.89 (d), 22.46 (s), 19.32 (s). GC/MS (EI): m/z (%): 353 (1) [M⁺], 294 (1), 220 (17), 185 (5), 176 (12), 135 (100), 129 (6), 105 (9), 71 (18).

### Example 27: (E)-3-(4-isobutyl-2-methylphenyl)-N-(1-(naphthalen-2-yl)ethyl)propan-1-imine oxide

The compound was obtained from 3-(4-isobutyl-2-methylphenyl)propanal (33.7 mmol, 7.24 g, 1.1 equiv) and N-(1-(naphthalen-2-yl)ethyl)hydroxylamine (30.6 mmol, 5.66 g, 1 equiv) according to the process of example 1 as white solid (18.4 mmol, 7.01 g, 60% yield).

¹H NMR (400 MHz, CDCl₃) δ 7.98-7.67 (m, 4H), 7.62-7.42 (m, 3H), 6.98-6.85 (m, 2H), 6.83-6.70 (m, 2H), 5.13 (q, J=6.7 Hz, 1H), 2.76 (s, 4H), 2.37 (d, J=7.1 Hz, 2H), 2.23 (s, 3H), 1.93-1.69 (m, 4H), 0.88 (d, J=6.5 Hz, 6H). ¹³C NMR (101 MHz, CDCl₃) b 139.73 (q), 137.15 (t), 135.77 (q), 135.73 (q), 135.65 (q), 133.34 (q), 133.16 (q), 131.14 (t), 128.59 (t), 128.28 (t), 128.22 (t), 127.67 (t), 126.73 (t), 126.65 (t), 126.51 (t), 126.40 (t), 125.03 (t), 73.63 (t), 44.98 (d), 30.20 (t), 28.24 (d), 26.77 (d), 22.47 (s), 19.33 (s), 19.12 (s). GC/MS (EI): m/z (%): 373 (1) [M⁺], 357 (3), 342 (3), 202 (17), 196 (7), 155 (100), 143 (5), 129 (10), 115 (9).

### Example 28: (E)-3-(4-isobutyl-2-methylphenyl)-N-(2-methylundecyl)propan-1-imine oxide

The compound was obtained from 3-(4-isobutyl-2-methylphenyl)propanal (28.6 mmol, 6.15 g, 1.1 equiv) and N-(2-methylundecyl)hydroxylamine (26.0 mmol, 5.18 g, 1 equiv) according to the process of example 1 as white solid (15.5 mmol, 6.01 g, 60% yield).

¹H NMR (400 MHz, CDCl₃) δ 7.07-7.00 (m, 1H), 6.96-6.86 (m, 2H), 6.64 (t, J=5.4 Hz, 1H), 3.66 (dd, J=11.8, 6.3 Hz, 1H), 3.43 (dd, J=11.8, 8.3 Hz, 1H), 2.93-2.70 (m, 4H), 2.40 (d, J=7.2 Hz, 2H), 2.31 (s, 4H), 1.89-1.77 (m, 1H), 1.37-1.21 (m, 16H), 0.92-0.84 (m, 12H). ¹³C NMR (101 MHz, CDCl₃) δ 139.79 (q), 138.77 (t), 135.75 (q), 135.71 (q), 131.20 (t), 128.13 (t), 126.79 (t), 71.74 (d), 44.99 (d), 34.06 (d), 31.91 (d), 30.79 (t), 30.20 (t), 29.78 (d), 29.62 (d), 29.59 (d), 29.34 (d), 28.28 (d), 26.65 (d), 22.69 (d), 22.43 (s), 19.34 (s), 17.04 (s), 14.13 (s). GC/MS (EI): m/z (%): 387 (1) [M⁺], 372 (1), 315 (1), 242 (3), 229 (4), 215 (7), 161 (100), 119 (49), 105 (22).

### Example 29: (E)-N-(sec-butyl)-3-(4-isobutyl-2-methylphenyl)propan-1-imine oxide

The compound was obtained from 3-(4-isobutyl-2-methylphenyl)propanal (25.5 mmol, 5.21 g, 1.1 equiv) and N-(sec-butyl)hydroxylamine (23.2 mmol, 2.00 g, 1 equiv) according to the process of example 1 as light yellow oil (11.9 mmol, 3.49 g, 51% yield).

¹H NMR (400 MHz, CDCl₃) δ 7.04 (d, J=7.5 Hz, 1H), 6.97-6.87 (m, 2H), 6.70 (t, J=5.3 Hz, 1H), 3.76-3.58 (m, 1H), 2.92-2.67 (m, 4H), 2.53-2.37 (m, 2H), 2.34-2.28 (m, 3H), 2.03-1.89 (m, 1H), 1.88-1.77 (m, 1H), 1.62-1.47 (m, 1H), 1.37 (t, J=10.4 Hz, 3H), 0.95-0.81 (m, 9H). ¹³C NMR (101 MHz, CDCl₃) δ 139.71 (q), 136.70 (t), 135.86 (q), 135.68 (q), 131.16 (t), 128.21 (t), 126.76 (t), 72.40 (t), 44.98 (d), 30.18 (t), 28.39 (d), 27.04 (d), 26.46 (d), 22.42 (s), 19.34 (s), 19.06 (s), 10.63 (s). GC/MS (EI): m/z (%): 275 (3) [M⁺], 246 (12), 187 (40), 161 (100), 131 (18), 119 (52), 105 (25), 57 (36).

### Example 30: (E)-3-(4-isobutyl-2-methylphenyl)-N-(1-phenylethyl)propan-1-imine oxide

The compound was obtained from 3-(4-isobutyl-2-methylphenyl)propanal (28.3 mmol, 5.78 g, 1.1 equiv) and N-(1-phenylethyl)hydroxylamine (25.7 mmol, 3.47 g, 1 equiv) according to the process of example 1 as light yellow oil (15.4 mmol, 5.26 g, 60% yield).

¹H NMR (400 MHz, CDCl₃) δ 7.44-7.28 (m, 5H), 6.94-6.80 (m, 3H), 6.72 (t, J=5.5 Hz, 1H), 5.03-4.82 (m, 1H), 2.86-2.64 (m, 4H), 2.40 (d, 2H), 2.31-2.20 (m, 3H), 1.89-1.71 (m, 4H), 0.89 (d, J=6.7 Hz, 6H). ¹³C NMR (101 MHz, CDCl₃) δ 139.71 (q), 138.36 (q), 137.02 (t), 135.84 (q), 135.66 (q), 131.17 (t), 128.73 (t), 128.65 (t), 128.62 (t), 128.34 (t), 127.40 (t), 126.80 (t), 73.46 (t), 45.04 (d), 30.24 (t), 28.25 (d), 26.80 (d), 22.50 (s), 19.35 (s), 19.07 (s). GC/MS (EI): m/z (%): 323 (43) [M⁺], 218 (19), 176 (24), 162 (29), 131 (29), 119 (33), 105 (100), 91 (31), 77 (29).

### Example 31: (Z)—N-cyclobutyl-3-(4-isobutyl-2-methylphenyl)propan-1-imine oxide

The compound was obtained from 3-(4-isobutyl-2-methylphenyl)propanal (31.0 mmol, 6.34 g, 1.1 equiv) and N-cyclobutylhydroxylamine (28.2 mmol, 2.40 g, 1 equiv) according to the process of example 1 as white solid (12.8 mmol, 3.69 g, 46% yield).

¹H NMR (400 MHz, CDCl₃) δ 7.10-6.99 (m, 1H), 6.98-6.83 (m, 2H), 6.72 (t, J=5.1 Hz, 1H), 3.63 (t, J=11.3 Hz, 1H), 2.84-2.75 (m, 3H), 2.40 (d, J=7.1 Hz, 2H), 2.30 (s, 3H), 2.05-1.73 (m, 7H), 1.66 (d, J=12.4 Hz, 1H), 0.89 (d, J=6.7 Hz, 6H). ¹³C NMR (101 MHz, CDCl₃) δ 139.75 (q), 136.19 (t), 135.86 (q), 135.72 (q), 131.17 (t), 128.22 (t), 126.77 (t), 73.82 (t), 44.98 (d), 31.04 (d), 30.22 (t), 28.36 (d), 26.57 (d), 25.06 (d), 25.00 (d), 22.45 (s), 19.38 (s). GC/MS (EI): m/z (%): 273 (1) [M⁺], 268 (3), 187 (31), 161 (55), 131 (32), 119 (100), 105 (25), 83 (9), 55 (55).

### Example 32: (E)-3-(4-isobutyl-2-methylphenyl)-N-(3-methylbut-2-en-1-yl)propan-1-imine oxide

The compound was obtained from 3-(4-isobutyl-2-methylphenyl)propanal (19.5 mmol, 3.99 g, 1.1 equiv) and N-(3-methylbut-2-en-1-yl)hydroxylamine (17.8 mmol, 2.44 g, 1 equiv) according to the process of example 1 as light yellow oil (4.5 mmol, 1.37 g, 26% yield).

¹H NMR (400 MHz, CDCl₃) δ 7.01 (d, J=7.5 Hz, 1H), 6.96-6.83 (m, 2H), 6.71 (t, J=5.3 Hz, 1H), 5.40 (t, J=7.2 Hz, 1H), 4.34 (d, J=7.4 Hz, 2H), 2.89-2.66 (m, 4H), 2.40 (d, J=7.1 Hz, 2H), 2.30 (s, 3H), 1.89-1.76 (m, 4H), 1.68 (s, 3H), 0.89 (d, J=6.6 Hz, 6H). ¹³C NMR (101 MHz, CDCl₃) δ 141.34 (q), 139.79 (q), 137.49 (t), 135.84 (q), 135.68 (q), 131.19 (t), 128.31 (t), 126.77 (t), 116.02 (t), 62.63 (d), 44.98 (d), 30.23 (t), 28.29 (d), 26.86 (d), 25.84 (s), 22.45 (s), 19.34 (s), 18.10 (s). GC/MS (EI): m/z (%): 287 (2) [M⁺], 219 (2), 207 (4), 187 (3), 161 (15), 131 (7), 119 (17), 105 (10), 69 (100).

### Example 33: (E)-N-isopropyldodecan-1-imine oxide

The compound was obtained from dodecanal (27.1 mmol, 5.01 g, 1 equiv) and N-isopropylhydroxylamine (15% aq. solution, 27.1 mmol, 13.58 g, 1 equiv) according to the process of example 9 as light yellow oil (14.5 mmol, 3.50 g, 53% yield).

¹H NMR (300 MHz, CDCl₃) δ 6.71 (t, J=5.7 Hz, 1H), 4.13-3.89 (m, 1H), 2.59-2.35 (m, 2H), 1.58-1.45 (m, 2H), 1.42 (d, J=6.5 Hz, 6H), 1.35-1.16 (m, 16H), 0.88 (t, J=5.7 Hz, 3H). ¹³C NMR (75 MHz, CDCl₃) δ 136.57 (t), 65.97 (t), 31.89 (d), 29.58 (d), 29.48 (d), 29.30 (d), 26.46 (d), 25.68 (d), 22.66 (d), 20.78 (s), 14.09 (s). GC/MS (EI): m/z (%): 241 (2) [M⁺], 226 (3), 198 (1), 128 (4), 114 (31), 101 (100), 86 (40), 72 (25), 55 (30).

### Example 34: (Z)-7-hydroxy-N-isopropyl-3,7-dimethyloctan-1-imine oxide

The compound was obtained from 7-hydroxy-3,7-dimethyloctanal (18.1 mmol, 3.13 g, 1 equiv) and N-isopropylhydroxylamine (15% aq. solution, 18.1 mmol, 9.08 g, 1 equiv) according to the process of example 9 as light yellow oil (13.2 mmol, 3.13 g, 73% yield).

¹H NMR (300 MHz, CDCl₃) δ 6.76 (t, J=5.8 Hz, 1H), 4.22-3.69 (m, 1H), 2.64-2.43 (m, 1H), 2.41-2.22 (m, 1H), 1.97-1.67 (m, 2H), 1.52-1.12 (m, 18H), 0.95 (d, J=6.7 Hz, 3H). ¹³C NMR (75 MHz, CDCl₃) δ 135.78 (t), 70.71 (q), 66.12 (t), 43.96 (d), 37.46 (d), 33.47 (d), 30.79 (t), 29.29 (s), 29.17 (s), 21.70 (d), 20.82 (s), 20.03 (s). GC/MS (EI): m/z (%): 229 (1) [M⁺], 214 (21), 196 (27), 156 (15), 128 (60), 112 (21), 101 (100), 86 (74), 59 (88).

### Example 35: (E)-3-(4-isobutylphenyl)-N-isopropyl-2-methylpropan-1-imine oxide

The compound was obtained from 3-(4-isobutylphenyl)-2-methylpropanal (48.9 mmol, 10.00 g, 1 equiv) and N-isopropylhydroxylamine (15% aq. solution, 48.9 mmol, 24.53 g, 1 equiv) according to the process of example 9 as white solid (10.4 mmol, 2.72 g, 21% yield).

¹H NMR (300 MHz, CDCl₃) δ 7.17-6.89 (m, 4H), 6.53 (d, J=7.2 Hz, 1H), 4.03-3.80 (m, 1H), 3.50-3.17 (m, 1H), 2.92-2.76 (m, 1H), 2.75-2.60 (m, 1H), 2.43 (d, J=7.1 Hz, 2H), 1.90-1.71 (m, 1H), 1.44-1.27 (m, 6H), 1.08 (d, J=6.9 Hz, 3H), 0.88 (d, J=6.6 Hz, 6H). ¹³C NMR (75 MHz, CDCl₃) δ 140.25 (t), 139.53 (q), 136.37 (q), 129.00 (t), 128.80 (t), 66.17 (t), 45.04 (d), 38.88 (d), 32.08 (t), 30.21 (t), 22.36 (s), 20.81 (s), 20.67 (s), 16.27 (s). GC/MS (EI): m/z (%): 261 (1) [M⁺], 246 (2), 187 (35), 147 (100), 131 (12), 105 (74), 91 (50), 72 (29), 57 (14).

### Example 36: (E)-N-isopropyl-2,6-dimethylhept-5-en-1-imine oxide

The compound was obtained from 2,6-dimethylhept-5-enal (15.4 mmol, 2.16 g, 1 equiv) and N-isopropylhydroxylamine (15% aq. solution, 15.4 mmol, 7.70 g, 1 equiv) according to the process of example 9 as light yellow oil (12.4 mmol, 2.57 g, 80% yield).

¹H NMR (300 MHz, CDCl₃) δ 6.42 (d, J=7.4 Hz, 1H), 4.94 (t, J=7.1 Hz, 1H), 3.96-3.66 (m, 1H), 3.06-2.86 (m, 1H), 1.95-1.62 (m, 2H), 1.51 (s, 3H), 1.46-1.40 (m, 3H), 1.37-1.17 (m, 8H), 0.92 (d, J=6.9 Hz, 3H). ¹³C NMR (75 MHz, CDCl₃) δ 140.81 (t), 131.54 (q), 123.75 (t), 65.91 (t), 33.95 (d), 30.21 (t), 25.53 (d), 25.50 (t), 20.67 (s), 20.53 (s), 17.41 (s), 16.28 (s). GC/MS (EI): m/z (%): 197 (24) [M⁺], 182 (100), 140 (27), 123 (30), 115 (10), 100 (9), 96 (9), 81 (24), 55 (15).

### Example 37: (E)-N-isopropyl-2-methyldecan-1-imine oxide

The compound was obtained from 2-methyldecanal (59.5 mmol, 10.12 g, 1 equiv) and N-isopropylhydroxylamine (15% aq. solution, 59.5 mmol, 29.80 g, 1 equiv) according to the process of example 9 as light yellow oil (41.6 mmol, 9.75 g, 70% yield).

¹H NMR (300 MHz, CDCl₃) δ 6.53 (d, J=7.5 Hz, 1H), 4.08-3.89 (m, 1H), 3.24-2.96 (m, 1H), 1.50-1.35 (m, 8H), 1.34-1.18 (m, 12H), 1.08 (t, J=6.6 Hz, 3H), 0.87 (t, J=6.5 Hz, 3H). ¹³C NMR (75 MHz, CDCl₃) δ 141.19 (t), 66.11 (t), 34.13 (d), 31.82 (d), 30.55 (t), 29.61 (d), 29.48 (d), 29.21 (d), 27.16 (d), 22.61 (d), 20.86 (s), 20.71 (s), 16.57 (s), 14.05 (s). GC/MS (EI): m/z (%): 227 (7) [M⁺], 212 (7), 185 (12), 170 (12), 142 (4), 128 (37), 115 (100), 100 (31), 86 (23).

### Example 38: (Z)-1-(4-(4-hydroxy-4-methylpentyl)cyclohex-3-en-1-yl)-N-isopropylmethanimine oxide (mixture of diastereoisomers)

The compound was obtained from 4-(4-hydroxy-4-methylpentyl)cyclohex-3-ene-1-carbaldehyde (16.8 mmol, 3.53 g, 1 equiv) and N-isopropylhydroxylamine (15% aq. solution, 16.8 mmol, 8.40 g, 1 equiv) according to the process of example 9 as colorless oil (9.2 mmol, 2.58 g, 55% yield).

¹H NMR (300 MHz, CDCl₃) δ 6.63 (d, J=7.1 Hz, 1H), 5.49-5.32 (m, 1H), 4.17-3.90 (m, 1H), 3.33-3.02 (m, 1H), 2.41-2.17 (m, 1H), 2.13-1.80 (m, 7H), 1.67-1.35 (m, 11H), 1.23 (s, 6H). ¹³C NMR (75 MHz, CDCl₃) δ 139.85 (t), 137.74 (q), 135.86 (q), 120.74 (t), 118.93 (t), 70.61 (q), 66.11 (t), 43.49 (d), 43.44 (d), 38.12 (d), 38.02 (d), 31.20 (t), 30.83 (t), 30.56 (d), 29.27 (t), 29.19 (t), 29.14 (t), 27.89 (d), 26.87 (d), 24.75 (d), 24.43 (d), 23.82 (d), 22.34 (d), 22.27 (d), 20.86 (s), 20.74 (s). GC/MS (EI): m/z (%): 267 (29) [M⁺], 252 (30), 232 (21), 180 (33), 150 (29), 117 (76), 105 (50), 91 (79), 59 (100).

### Example 39: (Z)-4-(4-hydroxyphenyl)-N-isopropylbutan-2-imine oxide (mixture of isomers)

The compound was obtained from 4-(4-hydroxyphenyl)butan-2-one (64.3 mmol, 10.56 g, 1 equiv) and N-isopropylhydroxylamine (15% aq. solution, 64.3 mmol, 32.20 g, 1 equiv) according to the process of example 9 as colorless oil (15.0 mmol, 3.52 g, 23% yield).

¹H NMR (300 MHz, CDCl₃) δ 9.70 (s, 1H), 7.07-6.89 (m, 2H), 6.87-6.73 (m, 2H), 4.52-3.57 (m, 1H), 2.93-2.59 (m, 4H), 2.19-1.91 (m, 3H), 1.43-1.14 (m, 6H). ¹³C NMR (75 MHz, CDCl₃) δ 156.56 (q), 155.87 (q), 149.71 (q), 149.26 (q), 130.69 (q), 129.24 (t), 129.16 (t), 116.05 (t), 115.66 (t), 57.59 (t), 57.23 (t), 36.09 (d), 35.46 (d), 32.29 (d), 29.62 (d), 19.79 (s), 19.62 (s), 19.10 (s), 18.91 (s). GC/MS (EI): m/z (%): 221 (1) [M⁺], 164 (42), 149 (8), 131 (4), 121 (14), 107 (100), 94 (14), 77 (24), 65 (7).

### Example 40: (E)-N-isopropyldec-9-en-1-imine oxide

The compound was obtained from dec-9-enal (97.0 mmol, 15.01 g, 1 equiv) and N-isopropylhydroxylamine (15% aq. solution, 97.0 mmol, 48.70 g, 1 equiv) according to the process of example 9 as light yellow oil (37.9 mmol, 8.00 g, 39% yield).

¹H NMR (300 MHz, CDCl₃) δ 6.73 (t, J=5.7 Hz, 1H), 5.94-5.32 (m, 1H), 4.98-4.83 (m, 1H), 4.14-3.88 (m, 1H), 2.48 (dd, J=13.4, 7.4 Hz, 2H), 2.03 (dd, J=13.1, 6.3 Hz, 2H), 1.65-1.09 (m, 17H). ¹³C NMR (75 MHz, CDCl₃) δ 138.96 (t), 136.48 (t), 114.11 (d), 65.87 (t), 33.66 (d), 29.34 (d), 29.05 (d), 28.86 (d), 28.75 (d), 26.37 (d), 25.56 (d), 20.70 (s). GC/MS (EI): m/z (%): 211 (2′) [M⁺], 196 (6), 154 (3), 114 (31), 101 (100), 86 (43), 72 (28), 69 (14), 59 (24).

### Example 41: (E)-N-isopropyl-3-(3-isopropylphenyl)butan-1-imine oxide

The compound was obtained from 3-(3-isopropylphenyl)butanal (52.6 mmol, 10.02 g, 1 equiv) and N-isopropylhydroxylamine (15% aq. solution, 52.6 mmol, 26.30 g, 1 equiv) according to the process of example 9 as light yellow oil (20.2 mmol, 5.01 g, 39% yield).

¹H NMR (300 MHz, CDCl₃) δ 7.33-7.16 (m, 1H), 7.12-6.95 (m, 3H), 6.56 (t, J=3.2 Hz, 1H), 4.07-3.78 (m, 1H), 3.18-2.96 (m, 1H), 2.95-2.60 (m, 3H), 1.41-1.03 (m, 15H). ¹³C NMR (75 MHz, CDCl₃) δ 149.15 (q), 145.66 (q), 135.15 (t), 128.49 (t), 125.10 (t), 124.43 (t), 124.11 (t), 66.02 (t), 36.83 (t), 34.45 (d), 34.13 (t), 24.04 (s), 22.59 (s), 20.75 (s), 20.69 (s). GC/MS (EI): m/z (%): 247 (2) [M⁺], 230 (2), 216 (2), 173 (67), 147 (100), 131 (28), 105 (61), 91 (50), 86 (19).

### Example 42: (E)-N-isopropyl-1-(naphthalen-2-yl)ethan-1-imine oxide

The compound was obtained from 1-(naphthalen-2-yl)ethan-1-one (58.8 mmol, 10.0 g, 1.1 equiv) and N-isopropylhydroxylamine hydrochloride (53.4 mmol, 5.96 g, 1 equiv) according to the process of example 9 as yellow oil (11.0 mmol, 2.50 g, 21% yield).

¹H NMR (300 MHz, CDCl₃) δ 8.02-7.79 (m, 3H), 7.75-7.64 (m, 1H), 7.62-7.49 (m, 2H), 7.42-7.17 (m, 1H), 4.67-4.37 (m, 1H), 2.50 (s, 3H), 1.37 (d, J=6.4 Hz, 6H). ¹³C NMR (75 MHz, CDCl₃) δ 145.33 (q), 133.92 (q), 133.01 (q), 132.99 (q), 129.05 (t), 128.17 (t), 127.83 (t), 127.18 (t), 127.06 (t), 126.54 (t), 124.46 (t), 59.15 (t), 20.80 (s), 20.38 (s). GC/MS (EI): m/z (%): 227 (1) [M⁺], 185 (55), 168 (30), 153 (27), 144 (40), 127 (100), 115 (50), 77 (22), 63 (30).

### Example 43: (E)-N-isopropyl-3-(2-methoxy-2-oxoethyl)-2-pentylcyclopentan-1-imine oxide (mixture of isomers)

The compound was obtained from methyl 2-(3-oxo-2-pentylcyclopentyl)acetate (110.0 mmol, 25.00 g, 1 equiv) and N-isopropylhydroxylamine (15% aq. solution, 110.0 mmol, 55.30 g, 1 equiv) according to the process of example 9 as yellow oil (17.4 mmol, 5.20 g, 16% yield).

¹H NMR (300 MHz, CDCl₃) δ 4.33-4.07 (m, 1H), 3.74-3.56 (m, 3H), 2.89-2.32 (m, 5H), 2.30-2.06 (m, 2H), 1.65-1.22 (m, 15H), 0.98-0.78 (m, 3H). ¹³C NMR (75 MHz, CDCl₃) δ 172.60 (q), 172.47 (q), 156.31 (q), 154.14 (q), 59.87 (t), 59.28 (t), 51.53 (s), 51.46 (s), 47.99 (t), 47.66 (t), 39.89 (t), 38.74 (d), 38.63 (d), 38.50 (t), 32.04 (d), 31.69 (d), 31.56 (d), 28.88 (d), 28.62 (d), 28.10 (d), 28.05 (d), 27.04 (d), 26.83 (d), 26.40 (d), 22.52 (d), 22.41 (d), 20.14 (s), 19.88 (s), 19.79 (s), 19.18 (s), 13.97 (s), 13.89 (s). GC/MS (EI): m/z (%): 283 (6) [M⁺], 252 (6), 212 (23), 181 (24), 153 (36), 140 (100), 124 (36), 99 (75), 81 (50).

### Example 44: (2E,3E)-N-benzyl-4-(2,6,6-trimethylcyclohex-1-en-1-yl)but-3-en-2-imine oxide (mixture of isomers)

The compound was obtained from β-Ionone (56.5 mmol, 10.87 g, 1.1 equiv) and N-benzylhydroxylamine hydrochloride (51.4 mmol, 8.10 g, 1 equiv) according to the process of example 1 as yellow oil (15.1 mmol, 4.50 g, 29% yield).

¹H NMR (400 MHz, CDCl₃) δ 7.47-7.16 (m, 5H), 6.73-6.30 (m, 2H), 5.18 (s, 2H), 2.24 (s, 3H), 2.06-1.92 (m, 2H), 1.85-1.37 (m, 7H), 1.02 (s, 6H). ¹³C NMR (101 MHz, CDCl₃) δ 145.51 (q), 137.23 (q), 135.96 (t), 134.45 (q), 133.35 (t), 132.36 (q), 128.87 (t), 128.84 (t), 128.09 (t), 127.69 (t), 127.36 (t), 125.01 (t), 124.14 (t), 63.99 (d), 39.50 (d), 34.16 (q), 33.22 (d), 29.02 (s), 28.87 (s), 21.61 (s), 18.99 (d), 13.50 (s). GC/MS (EI): m/z (%): 297 (6) [M⁺], 282 (10), 254 (3), 206 (3), 175 (2), 158 (8), 105 (10), 91 (100), 65 (9).

### Example 45: N-benzyl-4-((6-methylheptan-2-yl)oxy)butan-1-imine oxide

The compound was obtained from 4-((6-methylheptan-2-yl)oxy)butanal (25.8 mmol, 5.28 g, 1.1 equiv) and N-benzylhydroxylamine hydrochloride (23.5 mmol, 3.75 g, 1 equiv) according to the process of example 1 as light yellow oil (14.9 mmol, 4.61 g, 64% yield).

¹H NMR (400 MHz, CDCl₃) δ 7.43-7.32 (m, 5H), 6.81-6.63 (m, 1H), 4.87 (s, 2H), 3.56-3.42 (m, 1H), 3.40-3.27 (m, 2H), 2.65-2.52 (m, 2H), 1.82-1.67 (m, 2H), 1.57-1.41 (m, 2H), 1.36-1.24 (m, 3H), 1.19-1.12 (m, 2H), 1.10-1.04 (m, 3H), 0.86 (d, J=6.4 Hz, 6H). ¹³C NMR (101 MHz, CDCl₃) δ 139.41 (t), 133.00 (q), 129.28 (t), 128.90 (t), 128.87 (t), 75.59 (t), 69.14 (d), 67.82 (d), 39.05 (d), 36.82 (d), 27.92 (t), 26.13 (d), 24.32 (d), 23.30 (d), 22.64 (s), 22.61 (s), 19.62 (s). GC/MS (EI): m/z (%): 305 (1) [M⁺], 193 (1), 176 (3), 162 (3), 149 (8), 133 (10), 91 (100), 71 (21), 55 (7).

### Example 46: (1Z,4Z)—N-benzylhept-4-en-1-imine oxide

The compound was obtained from hept-4-enal (36.9 mmol, 4.14 g, 1.1 equiv) and N-benzylhydroxylamine hydrochloride (33.6 mmol, 5.36 g, 1 equiv) according to the process of example 1 as white solid (13.6 mmol, 3.03 g, 41% yield).

¹H NMR (400 MHz, CDCl₃) δ 7.49-7.29 (m, 5H), 6.60 (t, J=5.8 Hz, 1H), 5.48-5.34 (m, 1H), 5.31-5.19 (m, 1H), 4.88 (s, 2H), 2.64-2.46 (m, 2H), 2.24 (q, J=7.3 Hz, 2H), 2.06-1.85 (m, 2H), 0.92 (t, J=7.5 Hz, 3H). ¹³C NMR (101 MHz, CDCl₃) δ 138.80 (t), 133.34 (t), 132.88 (q), 129.36 (t), 128.93 (t), 126.99 (t), 69.23 (d), 26.74 (d), 23.04 (d), 20.51 (d), 14.24 (s). GC/MS (EI): m/z (%): 217 (1) [M⁺], 188 (2), 158 (1), 149 (2), 132 (2), 104 (3), 91 (100), 77 (4), 65 (18).

### Example 47: (Z)—N-benzyl-2-((3,7-dimethyloct-6-en-1-yl)oxy)ethan-1-imine oxide

The compound was obtained from 2-((3,7-dimethyloct-6-en-1-yl)oxy)acetaldehyde (21.4 mmol, 4.25 g, 1.1 equiv) and N-benzylhydroxylamine hydrochloride (19.5 mmol, 3.11 g, 1 equiv) according to the process of example 1 as light yellow solid (13.4 mmol, 4.27 g, 69% yield).

¹H NMR (400 MHz, CDCl₃) δ 7.40 (d, J=6.2 Hz, 5H), 6.80 (t, J=4.2 Hz, 1H), 5.14-5.03 (m, 1H), 4.88 (s, 2H), 4.40 (d, J=3.9 Hz, 2H), 3.48 (td, J=6.7, 3.7 Hz, 2H), 2.02-1.89 (m, 2H), 1.67 (s, 3H), 1.63-1.55 (m, 4H), 1.27 (d, J=8.1 Hz, 2H), 1.20-1.10 (m, 2H), 0.86 (d, J=6.6 Hz, 3H). ¹³C NMR (101 MHz, CDCl₃) δ 137.94 (t), 132.23 (q), 131.21 (q), 129.60 (t), 129.18 (t), 129.04 (t), 124.67 (t), 70.15 (d), 69.00 (d), 66.60 (d), 37.14 (d), 36.43 (d), 29.42 (t), 28.61 (q), 25.72 (t), 25.41 (d), 19.47 (s), 17.64 (s). GC/MS (EI): m/z (%): 303 (1) [M⁺], 234 (2), 207 (1), 164 (2), 145 (2), 120 (30), 91 (100), 69 (20), 55 (12).

### Example 48: (1Z,2E,6Z)—N-benzylnona-2,6-dien-1-imine oxide

The compound was obtained from nona-2,6-dienal (28.7 mmol, 4.26 g, 1.1 equiv) and N-benzylhydroxylamine hydrochloride (26.1 mmol, 3.97 g, 1 equiv) according to the process of example 1 as light yellow solid (11.5 mmol, 2.86 g, 44% yield).

¹H NMR (400 MHz, CDCl₃) δ 7.50-7.30 (m, 5H), 7.01 (d, J=9.4 Hz, 1H), 6.78 (dd, J=15.9, 9.4 Hz, 1H), 6.30-6.11 (m, 1H), 5.49-5.18 (m, 2H), 4.95-4.77 (m, 2H), 2.28-2.20 (m, 2H), 2.20-2.10 (m, 2H), 2.06-1.95 (m, 2H), 0.93 (t, J=7.5 Hz, 3H). ¹³C NMR (101 MHz, CDCl₃) δ 142.74 (t), 136.25 (t), 132.74 (t), 129.24 (t), 128.93 (t), 128.88 (t), 127.43 (t), 121.31 (t), 68.97 (d), 33.43 (d), 26.21 (d), 20.56 (d), 14.26 (s). GC/MS (EI): m/z (%): 243 (9) [M⁺], 214 (1), 184 (1), 160 (5), 121 (9), 106 (35), 91 (100), 79 (27), 65 (13).

### Example 49: (1Z,2E)-N-benzyldodec-2-en-1-imine oxide

The compound was obtained from dodec-2-enal (36.3 mmol, 66.20 g, 10% in TEC, 1.1 equiv) and N-benzylhydroxylamine hydrochloride (33.0 mmol, 5.27 g, 1 equiv) according to the process of example 1 as white solid (12.7 mmol, 3.69 g, 38% yield).

¹H NMR (400 MHz, CDCl₃) δ 7.47-7.29 (m, 5H), 6.99 (d, J=9.4 Hz, 1H), 6.81-6.68 (m, 1H), 6.28-6.10 (m, 1H), 4.88 (s, 2H), 2.19 (q, J=7.1 Hz, 2H), 1.69-1.65 (m, 1H), 1.47-1.36 (m, 2H), 1.30-1.20 (m, 11H), 0.88 (t, J=6.8 Hz, 3H). ¹³C NMR (101 MHz, CDCl₃) δ 143.83 (t), 136.41 (t), 133.05 (q), 129.30 (t), 128.96 (t), 128.90 (t), 120.98 (t), 68.94 (d), 33.37 (d), 31.88 (d), 29.50 (d), 29.43 (d), 29.30 (d), 29.19 (d), 28.61 (d), 22.68 (d), 14.12 (s). GC/MS (EI): m/z (%): 287 (1) [M⁺], 257 (1), 203 (21), 185 (2), 157 (100), 139 (4), 129 (8), 115 (35), 87 (8).

### Example 50: (1Z,2E)-N-benzyloct-2-en-1-imine oxide

The compound was obtained from oct-2-enal (31.7 mmol, 4.00 g, 1.1 equiv) and N-benzylhydroxylamine hydrochloride (28.8 mmol, 4.60 g, 1 equiv) according to the process of example 1 as white solid (25.4 mmol, 6.01 g, 88% yield).

¹H NMR (400 MHz, CDCl₃) δ 7.51-7.30 (m, 5H), 7.00 (d, J=9.4 Hz, 1H), 6.85-6.62 (m, 1H), 6.30-6.10 (m, 1H), 4.88 (s, 2H), 2.19 (q, J=7.2 Hz, 2H), 1.50-1.36 (m, 2H), 1.35-1.17 (m, 4H), 0.87 (t, J=6.7 Hz, 3H). ¹³C NMR (101 MHz, CDCl₃) δ 143.74 (t), 136.38 (t), 133.08 (q), 129.27 (t), 128.93 (t), 128.87 (t), 120.99 (t), 68.93 (d), 33.30 (d), 31.34 (d), 28.27 (d), 22.44 (d), 13.97 (s). GC/MS (EI): m/z (%): 231 (2) [M⁺], 215 (3), 188 (4), 172 (13), 160 (22), 144 (10), 104 (12), 91 (100), 65 (13).

### Example 51: (1Z,2E,6E)-N-benzyl-3,7-dimethylnona-2,6-dien-1-imine oxide (mixture of diastereoisomers)

The compound was obtained from 3,7-dimethylnona-2,6-dienal (10.8 mmol, 1.80 g, 1.1 equiv) and N-benzylhydroxylamine hydrochloride (9.8 mmol, 1.57 g, 1 equiv) according to the process of example 1 as light yellow oil (6.9 mmol, 1.90 g, 70% yield).

¹H NMR (400 MHz, CDCl₃) δ 7.50-7.31 (m, 5H), 7.26-7.17 (m, 1H), 6.61 (d, J=9.9 Hz, 1H), 5.10-4.95 (m, 1H), 4.90 (s, 2H), 2.26-2.04 (m, 4H), 2.03-1.88 (m, 3H), 1.77 (s, 2H), 1.69-1.63 (m, 1H), 1.62-1.51 (m, 2H), 0.99-0.90 (m, 3H). ¹³C NMR (101 MHz, CDCl₃) δ 150.17 (q), 150.02 (q), 149.95 (q), 138.49 (q), 138.31 (q), 138.09 (q), 137.85 (q), 133.88 (t), 133.65 (t), 133.41 (q), 133.34 (q), 129.05 (t), 128.87 (t), 128.76 (t), 122.79 (t), 122.50 (t), 121.67 (t), 121.28 (t), 116.74 (t), 116.72 (t), 115.90 (t), 69.11 (d), 69.05 (d), 40.70 (d), 40.43 (d), 33.89 (d), 33.62 (d), 32.32 (d), 32.29 (d), 26.39 (d), 26.21 (d), 26.15 (d), 25.93 (d), 24.77 (d), 24.74 (d), 24.50 (s), 24.47 (s), 22.85 (s), 18.00 (s), 15.94 (s), 15.90 (s), 12.88 (s), 12.81 (s), 12.76 (s), 12.74 (s). GC/MS (EI): m/z (%): 271 (6) [M⁺], 256 (2), 189 (12), 174 (33), 149 (17), 121 (16), 107 (14), 91 (100), 65 (12).

### Example 52: (Z)-1-(4-(4-hydroxy-4-methylpentyl)cyclohex-3-en-1-yl)-N-(2-methylundecyl)methanimine oxide

The compound was obtained from 4-(4-hydroxy-4-methylpentyl)cyclohex-3-ene-1-carbaldehyde (7.2 mmol, 1.50 g, 1.1 equiv) and N-(2-methylundecyl)hydroxylamine (6.5 mmol, 1.29 g, 1 equiv) according to the process of example 1 as light yellow oil (5.5 mmol, 2.20 g, 84% yield).

¹H NMR (400 MHz, CDCl₃) δ 6.54 (d, J=7.2 Hz, 1H), 5.48-5.32 (m, 1H), 3.71-3.60 (m, 1H), 3.47-3.37 (m, 1H), 3.28-3.14 (m, 1H), 2.38-2.24 (m, 2H), 2.14-2.04 (m, 1H), 2.01-1.79 (m, 6H), 1.65-1.57 (m, 1H), 1.51-1.41 (m, 5H), 1.30-1.23 (m, 13H), 1.23-1.16 (m, 8H), 0.97-0.82 (m, 6H). ¹³C NMR (101 MHz, CDCl₃) δ 143.29 (t), 137.78 (q), 137.73 (q), 118.94 (t), 71.80 (d), 70.92 (q), 43.47 (d), 38.05 (d), 33.93 (d), 31.90 (d), 31.05 (t), 30.75 (t), 29.79 (d), 29.59 (d), 29.34 (q), 29.21 (s), 27.87 (d), 26.87 (d), 26.60 (d), 24.86 (d), 22.70 (d), 22.31 (d), 16.93 (s), 14.15 (s). GC/MS (EI): m/z (%): 393 (1) [M⁺], 358 (9), 288 (8), 274 (78), 246 (17), 213 (35), 143 (35), 130 (100), 118 (51).

### Example 53: (1E)-N-cyclohexyl-9-hydroxy-5,9-dimethyldec-4-en-1-imine oxide

The compound was obtained from 9-hydroxy-5,9-dimethyldec-4-enal (18.3 mmol, 3.69 g, 1.1 equiv) and N-cyclohexylhydroxylamine (16.6 mmol, 1.88 g, 1 equiv) according to the process of example 1 as light yellow oil (12.9 mmol, 3.96 g, 77% yield).

¹H NMR (400 MHz, CDCl₃) δ 6.68 (t, J=5.5 Hz, 1H), 5.12 (m, 1H), 3.76-3.56 (m, 1H), 2.63-2.45 (m, 2H), 2.28-2.16 (m, 2H), 2.07-1.94 (m, 4H), 1.91-1.80 (m, 4H), 1.75-1.64 (m, 3H), 1.62-1.53 (m, 2H), 1.51-1.40 (m, 4H), 1.37-1.25 (m, 3H), 1.20 (s, 6H). ¹³C NMR (101 MHz, CDCl₃) δ 136.98 (q), 136.70 (q), 136.64 (t), 128.89 (t), 123.58 (t), 122.92 (t), 73.75 (t), 70.80 (q), 70.72 (q), 43.56 (d), 43.36 (d), 39.90 (d), 32.11 (d), 31.10 (d), 29.27 (s), 29.24 (s), 26.84 (d), 26.71 (d), 25.06 (d), 25.01 (d), 24.00 (d), 23.89 (d), 23.34 (s), 22.61 (d), 22.54 (d), 16.01 (s). GC/MS (EI): m/z (%): 295 (1) [M⁺], 280 (7), 208 (31), 192 (10), 178 (12), 141 (14), 122 (15), 95 (21), 55 (100).

### Example 54: (E)-N-benzyl-3-(3-isopropylphenyl)butan-1-imine oxide

The compound was obtained from 3-(3-isopropylphenyl)butanal (20.7 mmol, 3.93 g, 1.1 equiv) and N-benzylhydroxylamine hydrochloride (18.8 mmol, 3.01 g, 1 equiv) according to the process of example 1 as light yellow oil (16.6 mmol, 5.01 g, 88% yield).

¹H NMR (400 MHz, CDCl₃) δ 7.37-7.29 (m, 3H), 7.29-7.24 (m, 2H), 7.23-7.18 (m, 1H), 7.10-7.05 (m, 1H), 7.03-6.89 (m, 2H), 6.50 (t, J=5.8 Hz, 1H), 4.80 (s, 2H), 3.09-2.95 (m, 1H), 2.91-2.79 (m, 2H), 2.77-2.67 (m, 1H), 1.29 (d, J=7.0 Hz, 3H), 1.23 (d, J=7.0 Hz, 6H). ¹³C NMR (101 MHz, CDCl₃) δ 149.23 (q), 145.48 (q), 138.32 (t), 132.92 (q), 129.13 (t), 128.86 (t), 128.81 (t), 128.55 (t), 125.07 (t), 124.47 (t), 124.13 (t), 69.22 (d), 36.86 (t), 34.78 (d), 34.14 (t), 24.09 (s), 24.05 (s), 22.54 (s). GC/MS (EI): m/z (%): 295 (1) [M⁺], 279 (1), 264 (3), 173 (35), 147 (55), 131 (13), 105 (25), 91 (100), 65 (13).

### Example 55: (E)-N-benzyl-4-(4-hydroxyphenyl)butan-2-imine oxide

The compound was obtained from 4-(4-hydroxyphenyl)butan-2-one (102 mmol, 16.82 g, 1.1 equiv) and N-benzylhydroxylamine hydrochloride (93 mmol, 14.86 g, 1 equiv) according to the process of example 1 as colorless oil (25.8 mmol, 7.32 g, 28% yield).

¹H NMR (400 MHz, CDCl₃) δ 9.45 (s, 1H), 7.39-7.21 (m, 5H), 6.97-6.82 (m, 2H), 6.80-6.63 (m, 2H), 5.05 (d, J=17.3 Hz, 1H), 4.71 (s, 1H), 2.95-2.86 (m, 1H), 2.85-2.76 (m, 1H), 2.75-2.64 (m, 2H), 2.11 (s, 3H). ¹³C NMR (101 MHz, CDCl₃) δ 156.52 (q), 155.77 (q), 151.47 (q), 151.19 (q), 133.48 (q), 133.08 (q), 130.61 (q), 129.28 (t), 129.23 (t), 128.97 (t), 128.88 (t), 128.38 (t), 128.35 (t), 127.79 (t), 127.72 (t), 116.19 (t), 115.72 (t), 64.09 (d), 63.05 (d), 36.75 (d), 34.82 (d), 32.03 (d), 29.72 (d), 19.41 (s), 18.65 (s). GC/MS (EI): m/z (%): 269 (1) [M⁺], 253 (13), 238 (4), 162 (8), 146 (17), 121 (11), 107 (21), 91 (100), 77(23).

### Example 56: (E)-N-benzyldec-9-en-1-imine oxide

The compound was obtained from dec-9-enal (20.7 mmol, 3.19 g, 1.1 equiv) and N-benzylhydroxylamine hydrochloride (18.8 mmol, 3.01 g, 1 equiv) according to the process of example 1 as white solid (15.1 mmol, 4.01 g, 80% yield).

¹H NMR (400 MHz, CDCl₃) δ 7.53-7.31 (m, 5H), 6.63 (t, J=5.8 Hz, 1H), 5.93-5.61 (m, 1H), 5.13-4.68 (m, 4H), 2.58-2.38 (m, 2H), 2.10-1.95 (m, 2H), 1.57-1.43 (m, 2H), 1.41-1.19 (m, 8H). ¹³C NMR (101 MHz, CDCl₃) δ 139.48 (t), 139.07 (t), 133.03 (q), 129.25 (t), 128.91 (t), 128.87 (t), 114.21 (d), 69.17 (d), 33.73 (d), 29.40 (d), 29.08 (d), 28.91 (d), 28.81 (d), 26.73 (d), 25.50 (d). GC/MS (EI): m/z (%): 259 (1) [M⁺], 242 (3), 202 (1), 162 (5), 149 (30), 132 (9), 117 (5), 91 (100), 65 (15).

### Example 57: (Z)-1-(4-methoxyphenyl)-N-(2-methylundecyl)methanimine oxide

The compound was obtained from 4-methoxybenzaldehyde (11.5 mmol, 1.57 g, 1.1 equiv) and N-(2-methylundecyl)hydroxylamine (10.5 mmol, 2.09 g, 1 equiv) according to the process of example 1 as white solid (8.0 mmol, 2.64 g, 76% yield).

¹H NMR (400 MHz, CDCl₃) δ 8.28-8.18 (m, 2H), 7.26 (s, 1H), 6.97-6.88 (m, 2H), 3.89-3.73 (m, 4H), 3.64-3.47 (m, 1H), 2.57-2.25 (m, 1H), 1.44-1.19 (m, 16H), 1.00-0.93 (m, 3H), 0.91-0.82 (m, 3H). ¹³C NMR (101 MHz, CDCl₃) δ 160.96 (q), 134.29 (t), 130.48 (t), 123.51 (q), 113.83 (t), 73.14 (d), 55.35 (s), 34.14 (d), 31.89 (d), 31.32 (t), 29.76 (d), 29.61 (d), 29.57 (d), 29.32 (d), 26.73 (d), 22.68 (d), 17.16 (s), 14.12 (s). GC/MS (EI): m/z (%): 319 (10) [M⁺], 302 (100), 212 (14), 204 (12), 176 (7), 161 (7), 148 (67), 135 (37), 121 (54).

### Example 58: (E)-N-(4-methoxybenzyl)-2-methylundecan-1-imine oxide

The compound was obtained from 2-methylundecanal (12.7 mmol, 2.34 g, 1.1 equiv) and N-(4-methoxybenzyl)hydroxylamine (11.5 mmol, 1.74 g, 1 equiv) according to the process of example 1 as white solid (8.2 mmol, 2.62 g, 71% yield).

¹H NMR (400 MHz, CDCl3) δ 7.31 (d, J=8.5 Hz, 2H), 6.91 (d, J=8.5 Hz, 2H), 6.41 (d, J=7.5 Hz, 1H), 4.79 (s, 2H), 3.82 (s, 3H), 3.15-2.95 (m, 1H), 1.42-1.18 (m, 16H), 1.04 (d, J=6.9 Hz, 3H), 0.88 (t, J=6.8 Hz, 3H). ¹³C NMR (101 MHz, CDCl3) δ 160.03 (q), 143.86 (t), 130.77 (t), 125.17 (q), 114.31 (t), 68.80 (d), 55.32 (s), 34.03 (d), 31.90 (d), 30.96 (t), 29.60 (d), 29.54 (d), 29.32 (d), 27.17 (d), 22.69 (d), 16.50 (s), 14.12 (s). GC/MS (EI): m/z (%): 319 (1) [M⁺], 274 (1), 255 (3), 176 (2), 146 (5), 121 (100), 108 (4), 91 (14), 77 (35).

### Example 59: Application in Liquid Detergent

A Nitrone precursor (Sample A: (E)-2-methyl-N-(4-methylbenzyl)undecan-1-imine oxide, compound of Example 1) has been compared to the aldehyde (Sample B: 2-methyl undecanal) in a wash test.
1) Sample preparation: 0.2% by weight of the precursor or the aldehyde have been incorporated into unperfumed liquid detergent base by magnetic stirring at room temperature for 24 h.,
2) Wash test: A 40° C. machine wash cycle was performed using 55 g of the liquid detergent sample containing the test material and odour-neutral cotton/elastan mixed fabric T-shirts. The wet fabric is line-dried and odour intensity and quality are assessed by a panel of 4-6 experts on the fresh dried samples, after 1 and after 3 days on an intensity scale of 0 (odourless) to 5 (extremely strong).

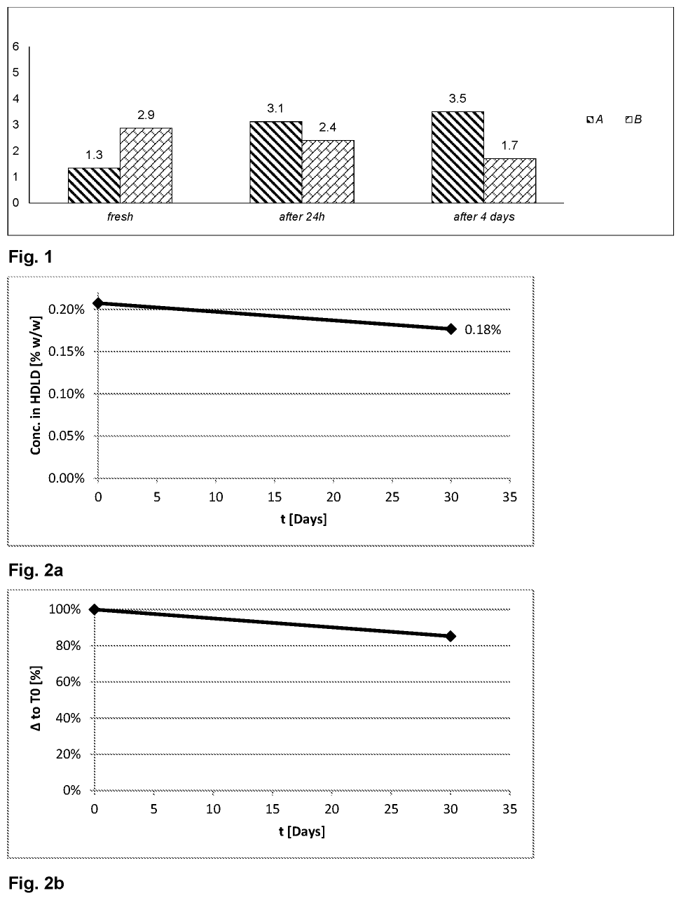

FIG. 1 shows the evaluation of the fresh dried samples (left), after 24 hours (middle) and after four days (right).

In sample B, the intensity of the aldehyde is relatively high on the freshly dried sample and decreases with time. For sample A, the intensity increases with time and is the highest after four days. This example clearly demonstrates the capability of the precursor to release the fragrance over an extended time period.

### Example 60: Further Application in Liquid Detergent

Further compounds of formula (I) have been tested in liquid detergent.
1) Sample preparation: 0.2% wt/wt of the test material was incorporated in unperfumed liquid detergent base by magnetic stirring at room temperature for 24 h.
2) Wash test: a 40° C. machine wash cycle was performed using 55 g of the liquid detergent sample containing the test material and odour-neutral cotton/elastane mixed fabric T-shirts. The wet fabric is line-dried and odour intensity and quality are assessed by a panel of 4-6 experts after 1 and 3 days on an intensity scale of 0 (odourless) to 5 (extremely strong). Table 1 shows the results of the assessment.

TABLE 1

| | | Fragrance | Fragrance | |
|---|---|---|---|---|
| Compound | | score | score | Odour |
| of | | after | after | on dry |
| example | Compound name | 1 day | 3 days | fabric |
| 5 | (E)-N-benzyl-2- | 3.2 | 2.9 | aldehydic |
| | methylundecan-1-imine | | | |
| | oxide | | | |
| 9 | (E)-N-isopropyl-2- | 2.4 | 3.2 | aldehydic, |
| | methylundecan-1-imine | | | fresh, |
| | oxide | | | incense |
| 10 | (2E,3E)-N-isopropyl- | 2.3 | 2.2 | ionone |
| | 4-(2,6,6- | | | |
| | trimethylcyclohex- | | | |
| | 1-en-1-yl)but-3-en-2- | | | |
| | imine oxide | | | |
| 49 | (1Z,2E)-N- | 2.9 | 3.0 | mandarin, |
| | benzyldodec-2-en-1- | | | citrus, |
| | imine oxide | | | coriander |

The results show that compounds of the present invention release noticeable fragrant odors on dry fabric in various perfumistic directions in liquid detergent application. The release of the fragrance takes place over an extended time period.

Example 61: Stability test in liquid detergentThe stability of (E)-N-isopropyl-2-methylundecan-1-imine oxide (compound of Example 9) in a liquid detergent base was assessed by quantitative HPLC with UV detection.

A liquid detergent sample as prepared above containing the test substance was sampled at TO and after 30 days storage at 37° C. (300 mg diluted with 10 mL acetonitrile/high purity water 1:1), and the concentration of the precursor was assessed by quantitative HPLC-UV using an external calibration curve. FIG. 2 shows the concentration (2a) and recovery (2b) over time.

After storage over one month, the initial concentration of the precursor decreased from 0.21 to 0.18 weight %. This means, that 85% of the precursor was still present in the liquid detergent. The substance was judged stable in liquid detergent, which, due to its composition and alkalinity, is regarded as a harsh chemical environment.

### Example 62: Application in Shampoo

1) Sample preparation: 0.2% wt/wt of (E)-N-isopropyl-2-methylundecan-1-imine oxide (compound of example 9) was incorporated in unperfumed shampoo base by magnetic stirring at room temperature for 24 h.
2) Wash test: Hair swatches were wetted with warm tap water, then 2 mL of the above prepared shampoo sample was applied to the hair swatches lathering with gloves delicately for 30 seconds. The hair swatches were rinsed with cold tap water for 20 seconds and excess water was removed by squeezing the hair swatches between two fingers. The hair swatches were left to dry in open air. Their odour intensity and quality was assessed by a panel of 4-6 experts after 1 and 2 days on an intensity scale of 0 (odourless) to 5 (extremely strong).
3) Results: After 1 day, the fragrance score was 3.2, after 2 days, the fragrance score was 3.3. The perceived odour was described as clean, aldehydic. The result shows that the compound of the present invention releases a noticeable fragrant odor on dry hair in shampoo application over extended time.

### Example 63: Assessment of Biodegradability

The results of the biodegradability assessment by the manometric respirometry test (OECD guideline for the testing of materials No. 301F, Paris 1992) are summarized in Table 2.

TABLE 2

| Compound | | | |
|---|---|---|---|
| of | | Biodegradation | |
| example | Compound name | in % | result |
| 5 | (E)-N-benzyl-2- | 87 | readily |
| | methylundecan-1-imine | | biodegradable |
| | oxide | | |
| 13 | (E)-N,2-dimethylundecan- | 89 | readily |
| | 1-imine oxide | | biodegradable |
| 15 | (E)-2-methyl-N-(2- | 75 | readily |
| | methylpentyl)undecan-1- | | biodegradable |
| | imine oxide | | |
| 1 | (E)-2-methyl-N-(4- | 83 | readily |
| | methylbenzyl)undecan-1- | | biodegradable |
| | imine oxide | | |
| 6 | (E)-N-cyclohexyl-2- | 74 | inherently |
| | methylundecan-1-imine | | biodegradable |
| | oxide | | |
| 58 | (E)-N-(4-methoxybenzyl)- | 76 | readily |
| | 2-methylundecan-1-imine | | biodegradable |
| | oxide | | |
| 9 | (E)-N-isopropyl-2- | 70 | inherently |
| | methylundecan-1-imine | | biodegradable |
| | oxide | | |

The results show that the compounds of the present invention are in general biodegradable. These results are surprising, since many examples of molecules with nitrogen-based functional groups are known to be not biodegradable.

A compound can be classified biodegradable, if it reaches the pass level of 60% oxygen consumption of theory required for complete mineralization.

It is readily biodegradable, if the pass level is reached within 10 days within the 28-day period of the test. The 10-day window begins when the degree of biodegradation has reached 10%.

If the pass level is obtained after 28-day period of the test, the compound can be classified as inherently biodegradable.

1. A method of utilizing a compound according to formula (I) 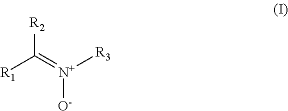
as precursor for generating a ketone or aldehyde of formula (II) 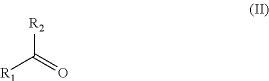
wherein
R₁ is selected from the group consisting of linear or branched C₁-C₁₅ alkyl;
linear or branched C₁-C₁₅ hydroxyalkyl, alkoxyalkyl and alkylthioalkyl;
linear or branched C₂-C₁₅ alkenyl;
linear or branched C₂-C₁₅ hydroxyalkenyl;
C₃-C₈ cycloalkyl;
C₃-C₈ cycloalkyl substituted with 1, 2, or 3 groups selected from C₁-C₇ alkyl, C₂-C₇ alkenyl, C₁-C₇ hydroxyalkyl, C2-C7 hydroxyalkenyl, and C3-C5 cycloalkyl;
C₅-C₈ cycloalkenyl;
C₅-C₈ cycloalkenyl substituted with 1, 2, or 3 groups selected from C₁-C₇ alkyl, C₂-C₇ alkenyl, C₁-C₇ hydroxyalkyl, C₂-C₇ hydroxyalkenyl, and C₃-C₅ cycloalkyl;
(C₁-C₃)alkyl(C₅-C₆)cycloalkyl;
(C₁-C₄)alkyl(C₅-C₆)cycloalkenyl;
(C₂-C₃)alkenyl(C₅-C₆)cycloalkenyl;
C₆-C₁₄ aryl;
C₆-C₁₄ aryl wherein the aryl ring is substituted with up to 3 groups selected from C₁-C₄ alkyl, —O—CH₂—O—, and —OR¹¹ wherein R¹¹ is independently selected from hydrogen and C₁-C₄ alkyl;
(C₁-C₄)alkyl(C₆-C₁₄)aryl;
(C₁-C₄)alkyl(C₆-C₁₄)aryl wherein the aryl-ring is substituted with up to 2 groups selected from C₁-C₄ alkyl, —O—CH₂—O—, and —OR¹² wherein R¹² is independently selected from hydrogen and C₁-C₄ alkyl;
(C₂-C₈)alkenyl(C₆-C₁₄)aryl;
(C₂-C₈)alkenyl(C₆-C₁₄)aryl wherein the aryl-ring is substituted with up to 2 groups selected from C₁-C₄ alkyl, —O—CH₂—O—, and —OR¹³ wherein R¹³ is independently selected from hydrogen and C₁-C₄ alkyl;
bi-, or tricyclic hydrocarbon ring comprising C₅-C₁₂ carbon atoms;
R₂ is selected from the group consisting of hydrogen, C₁-C₅ alkyl, and C₂-C₅ alkenyl;
or
R₁ and R₂ form together with the carbon atom to which they are attached a 5 to 16 membered hydrocarbon ring;
R₃ is selected from the group consisting of
linear or branched C₁-C₁₅ alkyl,
linear or branched C₂-C₁₅ alkenyl;
C₃-C₈ cycloalkyl,
bi-, or tricyclic hydrocarbon ring comprising C₅-C₁₂ carbon atoms,
and aromatic residues selected from p-isopropylbenzyl, benzyl, p-methylbenzyl, p-methoxybenzyl, 1-phenylethyl, benzo[d][1,3]dioxol-5-ylmethyl, naphthalen-2-ylmethyl; 1-(naphthalen-2-yl)ethyl; phenyl, p-nitrobenzyl; 3-ethoxy-4-hydroxybenzylpyridin-2-ylmethyl and furan-2-ylmethyl.

2. A compound according to formula (I) 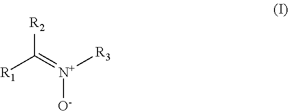
as precursor for generating a ketone or aldehyde of formula (II) 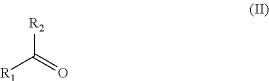
R₁ is selected from the group consisting of
linear or branched C₁-C₁₅ alkyl;
linear or branched C₁-C₁₅ hydroxyalkyl, alkoxyalkyl and alkylthioalkyl;
linear or branched C₂-C₁₅ alkenyl;
linear or branched C₂-C₁₅ hydroxyalkenyl;
C₃-C₈ cycloalkyl;
C₃-C₈ cycloalkyl substituted with 1, 2, or 3 groups selected from C₁-C₇ alkyl, C₂-C₇ alkenyl, C₁-C₇ hydroxyalkyl, C2-C7 hydroxyalkenyl, and C3-C5 cycloalkyl;
C₅-C₈ cycloalkenyl;
C₅-C₈ cycloalkenyl substituted with 1, 2, or 3 groups selected from C₁-C₇ alkyl, C₂-C₇ alkenyl, C₁-C₇ hydroxyalkyl, C₂-C₇ hydroxyalkenyl, and C₃-C₅ cycloalkyl;
(C₁-C₃)alkyl(C₅-C₆)cycloalkyl;
(C₁-C₄)alkyl(C₅-C₆)cycloalkenyl;
(C₂-C₃)alkenyl(C₅-C₆)cycloalkenyl;
C₆-C₁₄ aryl;
C₆-C₁₄ aryl wherein the aryl ring is substituted with up to 3 groups selected from C₁-C₄ alkyl, —O—CH₂—O—, and —OR¹¹ wherein R¹¹ is independently selected from hydrogen and C₁-C₄ alkyl;
(C₁-C₄)alkyl(C₆-C₁₄)aryl;
(C₁-C₄)alkyl(C₆-C₁₄)aryl, wherein the aryl-ring is substituted with up to 2 groups selected from C₁-C₄ alkyl —O—CH₂—O—, and —OR¹² wherein R¹² is independently selected from hydrogen and C₁-C₄ alkyl;
(C₂-C₈)alkenyl(C₆-C₁₄)aryl;
(C₂-C₈)alkenyl(C₆-C₁₄)aryl wherein the aryl-ring is substituted with up to 2 groups selected from C₁-C₄ alkyl, —O—CH₂—O—, and —OR¹³ wherein R¹³ is independently selected from hydrogen and C₁-C₄ alkyl;
bi-, or tricyclic hydrocarbon ring comprising C₅-C₁₂ carbon atoms;
R₂ is selected from the group consisting of hydrogen, C₁-C₅ alkyl, and C₂-C₅ alkenyl;
or
R₁ and R₂ form together with the carbon atom to which they are attached a 5 to 16 membered hydrocarbon ring;
R₃ is selected from the group consisting of
linear or branched C₁-C₁₅ alkyl,
linear or branched C₂-C₁₅ alkenyl;
C₃-C₈ cycloalkyl,
bi-, or tricyclic hydrocarbon ring comprising C₈-C₁₂ carbon atoms,
and aromatic residues selected from p-isopropylbenzyl, benzyl, p-methylbenzyl, p-methoxybenzyl, 1-phenylethyl, benzo[d][1,3]dioxol-5-ylmethyl, naphthalen-2-ylmethyl; 1-(naphthalen-2-yl)ethyl; phenyl, p-nitrobenzyl; 3-ethoxy-4-hydroxybenzylpyridin-2-ylmethyl and furan-2-ylmethyl;
with the proviso, that the compound is not 1-(4-hydroxy-3-methoxyphenyl)-N-isopropylmethanimine oxide, N-isopropyl-1-(4-methoxyphenyl)methanimine oxide, N-isopropylnonan-1-imine oxide, 1-(benzo[d][1,3]dioxol-5-yl)-N-isopropylmethanimine oxide, N-isopropyl-1-(p-tolyl)methanimine oxide, 1-(3,4-dimethoxyphenyl)-N-isopropylmethanimine oxide, N-isopropyl-2-methylpropan-1-imine oxide, N-isopropyldecan-1-imine oxide, N-isopropylhexan-1-imine oxide, 1-(furan-2-yl)-N-isopropylmethanimine oxide, N-benzyl-1-(4-methoxyphenyl)methanimine oxide, N-benzyldodecan-1-imine oxide, N-decyldecan-1-imine oxide.

3. A method to release a ketone or aldehyde of the formula (II) as defined in claim 1, wherein a compound of formula (I) as defined in claim 1 is exposed to oxygen and/or water.

4. A method of making a compound according to formula (I) as defined in claim 1, comprising the steps of:
a) converting a hydroxylamine into the corresponding hydrochloride, and
b) reacting the hydrochloride with a carbonyl compound to the compound of formula (I).

5. A consumer product comprising at least one compound according to formula I as defined in claim 1 and a product base.

6. The consumer product according to claim 5 wherein the consumer product is selected from home care and personal care products.

7. A method to confer, enhance, improve or modify the hedonic properties of a fragrance composition or a consumer product, wherein the method comprises adding to said composition or consumer product at least one compound of formula (I) as defined in claim 1.
